SEQUENCE LISTING (19 sequences)

SEQ ID NO: 1 - protein, 146 aa, synthetic construct
  REGION 1..146: note = Synthetic Construct
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 2 - protein, 223 aa, synthetic construct
  REGION 1..223: note = Synthetic Construct
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPASIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - protein, 228 aa, synthetic construct
  REGION 1..228: note = Synthetic Construct
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 4 - protein, 16 aa, synthetic construct
  REGION 1..16: note = Synthetic Construct
GSGGGSGGGGSGGGGS

SEQ ID NO: 5 - protein, 6 aa, synthetic construct
  REGION 1..6: note = Synthetic Construct
ASTKGP

SEQ ID NO: 6 - protein, 385 aa, synthetic construct
  REGION 1..385: note = Synthetic Construct
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIGSGGGSGGGGSGGGGSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPASIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 7 - protein, 375 aa, synthetic construct
  REGION 1..375: note = Synthetic Construct
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIASTKGPVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPASIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 8 - protein, 385 aa, synthetic construct [sequence omitted]

SEQ ID NO: 9 - protein, 375 aa, synthetic construct
  REGION 1..375: note = Synthetic Construct
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPASIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKASTKGPAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 10 - protein, 308 aa, synthetic construct
  REGION 1..308: note = Synthetic Construct
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIGSGGGSGGGGSGGGGSAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 11 - protein, 4 aa, synthetic construct
  REGION 1..4: note = Synthetic Construct
  VARIANT 1: note = Can be present in repeats of any integer
  VARIANT 2..4: note = Can be absent
GGGG

SEQ ID NO: 12 - protein, 4 aa, synthetic construct
  REGION 1..4: note = Synthetic Construct
  VARIANT 3..4: note = Can be absent or present in repeats of any integer
GSGS

SEQ ID NO: 13 - protein, 5 aa, synthetic construct
  REGION 1..5: note = Synthetic Construct
  VARIANT 1..5: note = Can be present in repeats of any integer
GSGGS

SEQ ID NO: 14 - protein, 5 aa, synthetic construct
  REGION 1..5: note = Synthetic Construct
  VARIANT 1..5: note = Can be present in repeats of any integer
GGGGS

SEQ ID NO: 15 - protein, 4 aa, synthetic construct
  REGION 1..4: note = Synthetic Construct
  VARIANT 1..4: note = Can be present in repeats of any integer
GGGS

SEQ ID NO: 16 - protein, 5 aa, synthetic construct
  REGION 1..5: note = Synthetic Construct
  VARIANT 1..5: note = Can be present in repeats of any integer up to 8
GGGGS

SEQ ID NO: 17 - protein, 15 aa, synthetic construct
  REGION 1..15: note = Synthetic Construct
GGGGSGGGGSGGGGS

SEQ ID NO: 18 - protein, 30 aa, synthetic construct
  REGION 1..30: note = Synthetic Construct
GGGGSGGGGSGGGGSGGGGSGGGGSGGGGS

SEQ ID NO: 19 - protein, 15 aa, synthetic construct
  REGION 1..15: note = Synthetic Construct
  VARIANT 1..15: note = Can be present in repeats of any integer up to 3
GSTSGSGKPGSGEGS

(19) United States
(12) Patent Application Publication
(10) Pub. No.: US 2026/0108616 A1
(43) Pub. Date: Apr. 23, 2026

(54) METHODS FOR TREATING GRAFT VERSUS HOST DISEASE

(71) Applicant: Evive Biotechnology (Shanghai) Ltd, Shanghai (CN)

(72) Inventors: William DALEY, Shanghai (CN); Xiaoqiang YAN, Shanghai (CN)

(21) Appl. No.: 19/426,688

(22) Filed: Dec. 19, 2025

Related U.S. Application Data
(63) Continuation of application No. 17/800,827, now Pat. No. 12,527,875.
(60) Provisional application No. 62/978,650, filed on Feb. 19, 2020.

(51) Int. Cl. A61K 47/64; A61K 31/573; A61P 37/06

(52) U.S. Cl. CPC A61K 47/642; A61K 31/573; A61P 37/06

(57) ABSTRACT

The present application provides methods for treating graft versus host disease (GvHD) by administering a composition an agent comprising an IL-22 (such as an IL-22 dimer), and optionally an immunosuppressive therapy. The treatment can be administered for multiple cycles which are separated by a rest period and/or individuals who have a specific Ann Arbor score or a characterized gut microbiota profile. The present application also provides methods of identifying or selecting individuals suitable for such treatments.

### CROSS REFERENCE TO RELATED APPLICATIONS

This application is a continuation of U.S. application Ser. No. 17/800,827, adopting international filing date of Feb. 19, 2021, which is a national stage application under 35 U.S.C. § 371 of International Application No. PCT/CN2021/076929, filed internationally on Feb. 19, 2021, which claims priority benefit of U.S. Provisional Application No. 62/978,650, filed on Feb. 19, 2020, the contents of each of which are incorporated herein by reference in their entireties.

### REFERENCE TO AN ELECTRONIC SEQUENCE LISTING

The contents of the electronic sequence listing (720622001701SEQLIST.xml; Size: 30,228 bytes; and Date of Creation: Dec. 12, 2025) is herein incorporated by reference in its entirety.

### FIELD OF THE INVENTION

This invention pertains to methods of treating graft vs. host disease (GvHD) and methods of identifying or selecting an individual having GvHD for treatment described herein.

### BACKGROUND OF THE INVENTION

Mechanisms regulating host tissue recovery from immune-mediated damage in graft vs. host disease (GvHD) remain incompletely understood. Current strategies to reduce clinical GvHD have the undesired effect of limiting both post-transplant immune function and therapeutic (beneficial) graft vs. leukemia/lymphoma (GVL) responses. For example, one concern is the maintenance and regeneration of intestinal epithelial tissues because GvHD may cause intestinal cell pathology which interferes with intestinal functions. GI GvHD is the predominant contributor to acute GvHD-related mortality after allogeneic hematopoietic stem/progenitor cell transplantation.

The disclosures of all publications, patents, patent applications and published patent applications referred to herein are hereby incorporated herein by reference in their entirety.

### BRIEF SUMMARY OF THE INVENTION

The present application provides methods of treating graft versus host disease (GvHD) in an individual, comprising administering to the individual: a) an effective amount of an IL-22 dimer comprising two monomeric subunits, wherein each monomeric subunit comprises an IL-22 domain and an Fc domain; and b) an immunosuppressive therapy. In some embodiments, the IL-22 dimer is administered for at least two cycles, wherein each of the at least two cycles comprises administering the IL-22 dimer for a period of at least two weeks, wherein two consecutive cycles of the at least two cycles are separated by a rest period of at least about one week. In some embodiments, the individual has an Ann Arbor score of 3, 2, and/or 1 prior to the treatment. In some embodiments, prior to the treatment: i) the fecal microbiota of the individual is characterized with a PC2 score of no more than 0.5 (such as no more than 0) based upon analysis of bacterial 16S rRNA sequencing of the fecal sample; and/or ii) the individual has a gut microbiota profile characterized with enriched Lachnospiraceae and Ruminococcaceae. In some embodiments, the individual has an infection. The present application also provides methods of identifying/selecting an individual for a treatment of graft verse host disease (GvHD) in the individual comprising: a) evaluating intestinal microbiome in the individual; and b) selecting an individual for treatment based upon: i) the individual has a gut microbiota profile characterized with enriched Lachnospiraceae and/or Ruminococcaceae; ii) the fecal microbiota of the individual is characterized with a PC2 score of no more than 0.5 (e.g., no more than 0) based upon analysis of bacterial 16S rRNA sequencing of the fecal sample; and/or iii) the individual has an Ann Arbor score of 3, wherein the treatment comprises: i) an effective amount of an IL-22 dimer comprising two monomeric subunits, wherein each monomeric subunit comprises an IL-22 domain and an Fc domain; and ii) an immunosuppressive therapy.

In one aspect of the present application, there is provided a method of treating graft versus host disease (GvHD) in an individual, comprising administering to the individual: a) an effective amount of an IL-22 dimer comprising two monomeric subunits, wherein each monomeric subunit comprises an IL-22 domain and an Fc domain; and b) an immunosuppressive therapy, wherein the IL-22 dimer is administered for at least two cycles, wherein each of the at least two cycles comprises administering the IL-22 dimer for a period of at least two weeks, wherein two consecutive cycles of the at least two cycles are separated by a rest period of at least about one week. In some embodiments, the IL-22 dimer is administered at a frequency of at least about once a week during each of the at least two cycles. In some embodiments, at least one of the at least two cycles comprises administering the IL-22 dimer at least about once a week for at least about four weeks. In some embodiments, each of the at least two cycles comprises administering the IL-22 dimer at least once a week for at least about four weeks. In some embodiments, at least one of the at least two cycles comprises administering the IL-22 dimer no more than about three months. In some embodiments, each of the at least two cycles comprises administering the IL-22 dimer no more than about three months. In some embodiments, the rest period is about one to four weeks. In some embodiments, the rest period is at least about four weeks. In some embodiments, the rest period is no more than three months. In some embodiments, the individual has an Ann Arbor score of 1, 2, and/or 3. In some embodiments, the fecal microbiota of the individual prior to the treatment is characterized with a PC2 score of no more than 0.5 based upon analysis of bacterial 16S rRNA sequencing of the fecal sample; or the individual has a gut microbiota profile characterized with enriched Lachnospiraceae and Ruminococcaceae.

In another aspect of the present application, there is provided a method of treating graft versus host disease (GvHD) in an individual, comprising administering to the individual: a) an effective amount of an IL-22 dimer comprising two monomeric subunits, wherein each monomeric subunit comprises an IL-22 domain and an Fc domain; and b) an immunosuppressive therapy, wherein the individual has an Ann Arbor score of 3 prior to the treatment. In some embodiments, the method further comprises assessing Ann Arbor score of the individual. In some embodiments, the method further comprises selecting the individual for the treatment based upon the individual having an Ann Arbor score of 3.

In another aspect of the present application, there is provided a method of treating graft versus host disease (GvHD) in an individual, comprising administering to the individual: a) an effective amount of an IL-22 dimer comprising two monomeric subunits, wherein each monomeric subunit comprises an IL-22 domain and an Fc domain; and b) an immunosuppressive therapy, and wherein prior to the treatment: i) the fecal microbiota of the individual is characterized with a PC2 score of no more than 0.5 based upon analysis of bacterial 16S rRNA sequencing of the fecal sample; or ii) the individual has a gut microbiota profile characterized with enriched Lachnospiraceae and Ruminococcaceae. In some embodiments, the method further comprises assessing bacterial 16s rRNA gene sequence in fecal sample of the individual. In some embodiments, the method further comprises selecting the individual for the treatment based upon the fecal microbiota of the individual having a PC2 score of no more than 0.5 based upon analysis of bacterial 16S rRNA sequencing of the fecal sample.

In another aspect of the present application, there is provided a method of treating graft versus host disease (GvHD) in an individual, comprising administering to the individual: a) an effective amount of an IL-22 dimer comprising two monomeric subunits, wherein each monomeric subunit comprises an IL-22 domain and an Fc domain; and b) an immunosuppressive therapy, wherein the individual has an infection. In some embodiments, the immunosuppressive therapy comprises one or more corticosteroids. In some embodiments, the one or more corticosteroids are contraindicated for the individual.

In another aspect of the present application, there is provided a method of identifying/selecting an individual for a treatment of graft verse host disease (GvHD) in the individual comprising: a) evaluating intestinal microbiome in the individual; and b) selecting an individual for treatment based upon: i) the individual has a gut microbiota profile characterized with enriched Lachnospiraceae and/or Ruminococcaceae; ii) the fecal microbiota of the individual is characterized with a PC2 score of no more than 0.5 based upon analysis of bacterial 16S rRNA sequencing of the fecal sample; and/or iii) the individual has an Ann Arbor score of 3, wherein the treatment comprises: i) an effective amount of an IL-22 dimer comprising two monomeric subunits, wherein each monomeric subunit comprises an IL-22 domain and an Fc domain; and ii) an immunosuppressive therapy. In some embodiments, the fecal microbiota of the individual prior to the treatment is characterized with a PC2 score of no more than 0 based upon analysis of bacterial 16S rRNA sequencing of the fecal sample.

In some embodiments according to any one of the methods described above, the method or the treatment comprises administering the IL-22 dimer for at least two cycles, wherein each of the at least two cycles comprises administering the IL-22 dimer at least about once a week for a period of at least two weeks, wherein two consecutive cycles of the at least two cycles are separated by a rest period of at least about one week.

In some embodiments according to any one of the methods described above, the IL-22 domain comprises a recombinant IL-22. In some embodiments, said recombinant IL-22 is a human IL-22. In some embodiments, the IL-22 dimer comprises the amino acid sequence of any one of SEQ ID NOS: 6-9. In some embodiments, the IL-22 dimer comprises the amino acid sequence of SEQ ID NO: 6.

In some embodiments according to any one of the methods described above, the amount of the IL-22 dimer for each administration is about 1 μg/kg to about 500 μg/kg for a human being or a comparable dose for an individual who is not a human being. In some embodiments, the amount of the IL-22 dimer for each administration is about 2 μg/kg to about 200 μg/kg for a human being or a comparable dose for an individual who is not a human being. In some embodiments, the amount of the IL-22 dimer for each administration is about 5 μg/kg to about 80 μg/kg, or about 10 μg/kg to about 45 μg/kg (e.g., 10 μg/kg, 30 μg/kg, or 45 μg/kg) for a human being or a comparable dose for an individual who is not a human being. In some embodiments, the amount of the IL-22 dimer for each administration is about 45 μg/kg for a human being or a comparable dose for an individual who is not a human being.

In some embodiments according to any one of the methods described above, the IL-22 dimer is administered intravenously.

In some embodiments according to any one of the methods described above, the GvHD is acute GvHD.

In some embodiments according to any one of the methods described above, the GvHD is a gastrointestinal GvHD (GI GvHD). In some embodiments, the GI GvHD is lower GI GvHD. In some embodiments, the lower GI GvHD is selected from the group consisting of Grade II, Grade III and Grade IV lower GI GvHD.

In some embodiments according to any one of the methods described above, the individual has not been subject to corticosteroid for GvHD for a period of three or more days prior to the treatment.

In some embodiments according to any one of the methods described above, the individual does not have an ongoing Cytomegalovirus (CMV) infection immediately prior to the treatment.

In some embodiments according to any one of the methods described above, the individual is a human.

In some embodiments according to any one of the methods described above, the immunosuppressive therapy comprises a corticosteroid drug. In some embodiments, the corticosteroid drug is a systemic corticosteroid. In some embodiments, the corticosteroid drug is a prednisone. In some embodiments, the prednisone is administered at a dose of no more than about 2 mg/kg for a human being or a comparable dose for an individual who is not a human being. In some embodiments, the corticosteroid drug is administered daily.

### BRIEF DESCRIPTION OF THE DRAWINGS

FIG. 1 depicts response rates at Day 28 and Day 56 after treatment.

FIG. 2 depicts response rates in patients with high, intermediate, or low Ann Arbor GvHD score (i.e., the score of 3, 2, or 1).

FIGS. 3A-3B depict H&E stained histopathologic assessment performed at the onset of aGvHD (pre-treatment) and after IL-22 treatment. FIG. 3A depicts results of a colonic biopsy with crypt injury and extensive epithelial cell apoptosis. Arrows point to apoptotic cells. FIG. 3B depicts results of a colonic biopsy Day 28 after treatment, which shows colonic mucosa with only rare epithelial apoptotic bodies in basal crypts. Arrow points to a single apoptotic cell.

FIGS. 4A-4B depict intestinal microbiota diversity according to treatment response (n=17) (FIG. 4A) and relative abundance of Genus Blautia according to treatment response (n=17) (FIG. 4B).

FIGS. 5A-5B depict principal analysis of 16S-sequenced Bray-Curtis distances of fecal samples collected at baseline (FIG. 5A) and peri-day 28 (post-treatment, FIG. 5B). Post-treatment samples from responding patients are segregated along PC2, indicating a global difference in microbiota composition.

### DETAILED DESCRIPTION OF THE INVENTION

The present application provides method of treating graft versus host disease (GvHD) in an individual, comprising administering to the individual an effective amount of an agent comprising IL-22 (such as an IL-22 dimer). In some embodiments, the method comprises administering to the individual a) an effective amount of an IL-22 dimer comprising two monomeric subunits, wherein each monomeric subunit comprises an IL-22 domain and an Fc domain, and b) an immunosuppressive therapy, wherein the IL-22 dimer is administered for at least two cycles, wherein each of the at least two cycles comprises administering the IL-22 dimer for a period of at least two weeks, wherein two consecutive cycles of the at least two cycles are separated by a rest period of at least about one week. In some embodiments, the method comprises administering to the individual: a) an effective amount of an IL-22 dimer comprising two monomeric subunits, wherein each monomeric subunit comprises an IL-22 domain and an Fc domain; and b) an immunosuppressive therapy, wherein the individual has an Ann Arbor score of 3 prior to the treatment. In some embodiments, the method comprises administering to the individual: a) an effective amount of an IL-22 dimer comprising two monomeric subunits, wherein each monomeric subunit comprises an IL-22 domain and an Fc domain; and b) an immunosuppressive therapy, and wherein prior to the treatment: i) the fecal microbiota of the individual is characterized with a PC2 score of no more than 0.5 based upon analysis of bacterial 16S rRNA sequencing of the fecal sample; or ii) the individual has a gut microbiota profile characterized with enriched Lachnospiraceae and Ruminococcaceae. In some embodiments, the method comprises administering to the individual: a) an effective amount of an IL-22 dimer comprising two monomeric subunits, wherein each monomeric subunit comprises an IL-22 domain and an Fc domain; and b) an immunosuppressive therapy, wherein the individual has an infection. The present application also provides methods of identifying/selecting an individual for treatment described herein. In some embodiments, the method comprises a) evaluating intestinal microbiome in the individual; and b) selecting an individual for treatment based upon: i) the individual has a gut microbiota profile characterized with enriched Lachnospiraceae and/or Ruminococcaceae; ii) the fecal microbiota of the individual is characterized with a PC2 score of no more than 0.5 based upon analysis of bacterial 16S rRNA sequencing of the fecal sample; or iii) the individual has an Ann Arbor score of 3.

The present application is at least partly based upon the surprisingly advantageous preliminary results from a Phase Ila study on the effect of a representative agent comprising IL-22 (i.e., F-652, an IL-22 dimer) on treating GvHD. As described in Example 1 in more details, patients having GvHD and treated with F-652 achieved unexpected durable response that prolongs at least about four weeks after completion of the treatment. Such results provide strong support for a regimen that has multiple cycles separated by rest period to better toxicity management. Moreover, patients with low Ann Arbor score (Ann Arbor score=1) achieved 100% response rate; patients with intermediate Ann Arbor score (Ann Arbor score=2) achieved 75% response rate; and patients with high Ann Arbor score (Ann Arbor score=3), a population very difficult to treat, achieved a response rate of about 58%. Such high response rates in all three populations, especially the population with high Ann Arbor score, are striking. Moreover, the preliminary study also provides strong evidence that patients treated with F-652 achieved a) a more diverse and/or abundant gut microbiota profile. Furthermore, the results also suggest that patients with a gut microbiota profile characterized with enriched Lachnospiraceae and/or Ruminococcaceae or having a fecal microbiota characterized with a PC2 score of no more than 0.5 based upon analysis of bacterial 16S rRNA sequencing of the fecal sample responds more favorably to the treatment described herein.

### Definitions

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

As used herein, the term “IL-22 polypeptide” or “IL-22” or “IL22” or “IL-22 protein” refers to a biologically active polypeptide capable of producing the biological activity as described herein. IL-22 of the present invention includes but not limited to human IL-22, recombinant human IL-22, murine IL-22 and/or recombinant murine IL-22. Specific polypeptide sequences are described in U.S. Patent Pub. No. US2003/0100076, U.S. Pat. Nos. 7,226,591 and 6,359,117, herein incorporated by reference in their entirety. “IL-22” also includes modified IL-22, such as pegylated IL-22 and covalently modified IL-22 proteins. The IL-22 polypeptides used herein may be isolated from a variety of sources, such as from human tissue types or from another source, or prepared by recombinant or synthetic methods. For example, descriptions of the preparation of, purification of, derivation of, formation of antibodies to or against, administration of, compositions containing, treatment of a disease with, etc., pertain to each polypeptide of the invention individually. Additionally, the IL-22 for use in the present inventions may be a product of a recombinant method wherein the IL-22 encoding DNA is administered to a subject, for example, such as lactobacilli expressing IL-22. The term “IL-22 polypeptide” also includes variants of the IL-22 polypeptides. The IL-22 of the present invention may also be modified in a way to form a chimeric molecule comprising IL-22 fused to another, heterologous polypeptide or amino acid sequence. The term IL-22 as used herein, includes both a monomer and a dimer form of IL-22 (such as IL-22 dimer as described herein). IL-22 is also known as interleukin-10 related T cell-derived inducible factor (IL-TIF).

As used herein, the term “IL-22 monomer” refers to one unit of an IL-22 protein.

As used herein, the term “IL-22 dimer” refers to a protein having more than one unit of an IL-22 molecule, for one example, an IL-22 dimer may have two IL-22 molecules linked together using linkers such as a short polypeptide, a chemical bond, and a covalent bond. In some embodiments, an IL-22 dimer contains two duplicate IL-22 molecules, in other embodiments, an IL-22 dimer is made up of different IL-22 proteins. Further examples of IL-22 dimers that may find use in the present inventions are described in U.S. patent application No. 20130171100, herein incorporated by reference in its entirety. One suitable IL-22 dimer is a recombinant IL-22 dimerized protein containing human interleukin 22 (IL-22) and produced in transformed Chinese Hamster Ovary (CHO) cells in serum-free culture produced by Generon (Shanghai) Corporation Ltd (now Evive Biotechnology (Shanghai) Ltd). IL-22 dimers are described, for example, in U.S. patent application No. 20130171100, including sequence information, herein incorporated by reference in its entirety. IL-22 dimer forming polypeptides used herein may be isolated from a variety of sources, such as from human tissue types or from another source, or prepared by recombinant or synthetic methods. In some embodiments, an IL-22 dimer comprises a carrier protein, including but not limited to an Fc fragment of human IgG (1, 2, 3, 4), or human albumin. IL-22 can be localized at the C-terminal or N-terminal of the carrier protein.

As used herein, the term “graft-versus-leukemia” or “GVL” or “Graft-versus-tumor effect” or “GVT” refers to a beneficial therapeutic immune reaction of the grafted donor T lymphocytes against the diseased bone marrow and residual tumor cells of the recipient. As used herein, the term “gastrointestinal graft vs. host disease” and “GI-GvHD” refers to damage caused by donor immune cells to host tissue of the stomach and intestine which can cause loss of appetite, nausea, vomiting, or diarrhea as part of GvHD, either acute or chronic. In severe cases, GI-GvHD can cause pain in the abdomen and bleeding in the stomach or intestines.

As used herein, the term “allogeneic transplant” refers to donor blood infusions or marrow stem cell transplants from a donor to a host patient. In other words, the patient receives bone marrow or blood stem cells from a tissue-matched or a close matched donor, i.e. matched at major HLA loci, who may or may not be a relative. Identical twin allogeneic transplants are called syngeneic transplants.

As used herein, the term “hematopoietic stem cell transplantation” or “HSCT” or “hematopoietic cell transplantation” or “HCT” refers to a transplantation of multipotent hematopoietic cells, including stem cells, usually derived from bone marrow, peripheral blood, or umbilical cord blood.

As used herein, the term “progenitor cell” in reference to an intestinal cell refers to multipotent cells that may give rise to differentiated cells of the small or large intestine, such as a columnar cell and a goblet cell.

As used herein, the term “organoid” in reference to an intestinal organoid comprising a central lumen lined by a villus-like epithelium that result from culturing of epithelial stem cells or isolated crypts in a culture medium. Crypts refer to intestinal stem/progenitor cell niche at the base of the epithelium.

As used herein, “cells” refer to the structural unit of an organism consisting of a nucleus and organelles surrounded by a semipermeable cell membrane. It is not intended to be limited to live or functioning cells.

As used herein, the term “contacting” or “treating” or “administering” a compound to a cell or tissue, such as an IL-22 protein, or IL-22 protein composition, or cytokine, or cytokine composition and the like, refers to placing the compound in a location that will allow it to touch the cell in order to produce “contacted” or “treated” cells. The contacting may be accomplished using any suitable method. For example, in one embodiment, contacting is by adding the compound to a tube of cells. Contacting may also be accomplished by adding the compound to cells in a microtiter plate. Contacting may also be accomplished by adding the compound to a culture of the cells or to an organoid culture. It is not meant to limit how the compound contacts the cells. In one embodiment, contacting may be accomplished by administration of the compound, such as an IL-22 molecule composition to an animal in vivo.

As used herein, the terms “culture media,” and “cell culture media,” refers to media that are suitable to support the growth of cells in vitro (i.e., cell cultures). It is not intended that the term be limited to any particular cell culture medium. For example, it is intended that the definition encompass outgrowth as well as maintenance media. Indeed, it is intended that the term encompass any culture medium suitable for the growth of the cell cultures of interest.

The term “sample” such as a “test sample” is used in its broadest sense. In one sense it can refer to an animal cell or tissue including a human cell or tissue. In another sense, it is meant to include a specimen or culture obtained from any source, in particular as a biological sample. Biological samples may be obtained from animals (including humans) and encompass fluids, solids, gases, tissues, cells, blood bone marrow and bones.

As used herein, the term “portion” when used in reference to a population of cells (as in “a portion of intestinal cells” or “a portion of bone marrow cells”) refers to at least one cell of that population up to 99% of those cells. For example, where contacting results in at least a “portion” of said cell population, it should be clear that portion is with reference to a population.

As used herein, “polypeptide” or “protein” refers to an amino acid, amino acid sequence, oligopeptide, peptide, or protein or portions thereof whether naturally occurring or synthetic.

As used herein, the term “portion” when used in reference to a protein (as in “a portion of a given protein”) refers to fragments of that protein. The fragments may range in size from four amino acid residues to the entire amino sequence minus one amino acid.

As used herein, the term “altered function” refers to a change in function, either increasing a function or decreasing a function, for example, a change in cell numbers, such as total thymocytes, a change in cell type, such as a change in the number of pre B cells, a change in the number of CD8+ cells, a change in function, such as epithelial cells capable of secreting a specific cytokine or inducing survival or maturation of a specific cell type, and the like.

As used herein, the term “in vitro assay” refers to any in vitro assay used to measure the increase or decrease of function or number of cells or cell subtypes. Readouts for in vitro assays of organoid cultures could include for examples, flow cytometry measurements of intestinal stem cell progeny, such as basal crypt cells. Readouts for in vitro assays of immune function could include for examples, T cell functional assays, including cytokine production, T cell subtypes, granulocytes, stromal cells, proliferation, extent of apoptosis, etc., see assays used in the Examples.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein, particularly in reference to a “human subject.” For the purposes of the present inventions, a subject may be immunocompromised, i.e. not able to fight off infections or control abnormal cell growth. Examples of immunocompromised subjects include subjects that have any of the following conditions, chemotherapy, exposure to radiation, deliberate irradiation, human immunodeficiency virus infections, transplantation, etc.

The terms “treatment”, “treating” and the like are used herein to generally mean obtaining a desired pharmacological and/or physiological effect. In relation to a therapeutic treatment of subject the effect may be prophylactic in terms of completely or partially preventing a disease or symptom thereof and/or may be therapeutic in terms of partially or completely curing a disease and/or adverse effect attributed to the disease. Thus “treatment” refers to both therapeutic treatment and prophylactic or preventative measures, wherein the object is to prevent or slow down (lessen) the targeted pathologic condition or disorder. Subjects in need of treatment include those already with the disorder as well as those prone to have the disorder or those in whom the disorder is to be prevented.

As used herein, when referring to a method of the present invention the term “treatment” covers any treatment of a disease in a mammal, particularly a human, and includes: (a) preventing the disease from occurring in a subject which may be predisposed to the disease but has not yet been diagnosed as having it; (b) inhibiting the disease, i.e. arresting its development; or (c) relieving the disease, i.e. causing regression of the disease. The present invention is directed towards treating patients with medical conditions relating to a loss of immunocompetence from a treatment related to a disease such as irradiation, chemotherapy, immunosuppression, etc. Accordingly, a treatment of the invention would involve preventing, inhibiting or relieving any medical condition where a desired level of immunocompetence would be achieved by the use of an IL-22 composition of the present inventions. In certain embodiments, treatment refers to exposing a subject to a therapy directed towards treating a disease, such as irradiation, chemotherapy, and the like.

As used herein, the term “administering” or “administration” refers to the act of giving a drug, prodrug, pharmaceutical composition, or other agent, or therapeutic treatment (e.g., a composition of the present invention) to a physiological system (e.g., a subject or in vivo, in vitro, or ex vivo cells, tissues, and organs). Acceptable routes of administration to the human body can be through the eyes (ophthalmic), mouth (oral), skin (transdermal), nose (nasal), lungs (inhalant), mucosal (e.g., oral mucosa or buccal), rectal, ear, by injection (e.g., intravenously, subcutaneously, intratumorally, intraperitoneally, etc.) and the like. Administration “in combination with” one or more further therapeutic agents includes simultaneous (concurrent) and consecutive administration in any order.

As used herein, the term “pharmaceutical” or “therapeutic” in reference to a composition refers to the combination of an active agent (for example, in an effective amount) (e.g., such as an IL-22 protein or IL-22 DNA)) with a carrier, inert or active, making the composition especially suitable for diagnostic or therapeutic use in vitro, in vivo or ex vivo (in vitro).

An “effective amount” of a polypeptide disclosed herein or an agonist or antagonist thereof is an amount sufficient to carry out a specifically stated purpose. An “effective amount” may be determined empirically and in a routine manner, in relation to the stated purpose. Examples of effective amounts in using the pharmaceutical composition, a safe and effective amount of the IL-22 dimer of the present invention is administrated to a mammal (e.g. human) in need thereof, in which the dosage administrated is a pharmaceutically acceptable effective administration dosage. As one example, for a human of 60 kg, the administration dosage is usually 0.01-300 mg; in a preferred embodiment, the administration dosage is 0.5-100 mg, see, for example, U.S. patent application No. 20130171100, herein incorporated by reference in its entirety.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintegrants (e.g., potato starch or sodium starch glycolate), and the like. The compositions also may include stabilizers and preservatives. Examples of carriers, stabilizers, and adjuvants are described in the art (See e.g., Martin, Remington's Pharmaceutical Sciences, 15th Ed., Mack Publ. Co., Easton, Pa. (1975), incorporated herein by reference).

The terms “pharmaceutically acceptable” or “pharmacologically acceptable,” as used herein, refer to compositions that do not substantially produce adverse reactions (e.g., toxic, allergic, or immunological reactions) when administered to a subject. Examples of forms of IL-22 of the present invention used for administration comprise ointment, powder, patch, sprayer, and inhalant.

As used herein, “cytokine” refers to a protein or glycoprotein that is used in an organism as signaling compounds. It is intended to include homologues and synthetic versions. Examples include IL-22, IL-23, IL-21, the IL-10 family, IL-7, the interferon (IFN) family, CC chemokines, CXC chemokines, and the like.

As used herein the term “biologically active polypeptide” refers to any polypeptide which maintains a desired biological activity, for one example, biological IL-22 activities as described herein, such as increasing intestinal stem cell and intestinal progenitor cell division, maturation, and the like.

Where “amino acid sequence” is recited herein to refer to an amino acid sequence of a naturally occurring protein molecule, “amino acid sequence” and like terms, such as “polypeptide” and “protein” are not meant to limit the amino acid sequence to the complete, native amino acid sequence associated with the recited protein molecule. As used herein the term “recombinant protein” or “recombinant polypeptide” refers to a protein molecule that is expressed from a recombinant DNA molecule (e.g. human IL-22 expressed by cells containing a plasmid or virus expressing a human IL-22 gene).

As used herein the term “recombinant DNA molecule” refers to a DNA molecule that is comprised of segments of DNA joined together by means of molecular biological techniques (e.g. a human IL-22 gene ligated into a plasmid DNA sequence or viral sequence).

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

The term “gene” refers to a nucleic acid (e.g., DNA) sequence that comprises coding sequences necessary for the production of a polypeptide or precursor. It is intended that the term encompass polypeptides encoded by a full length coding sequence, as well as any portion of the coding sequence, so long as the desired activity and/or functional properties (e.g., IL-22 activity, ligand binding, enzymatic activity, etc.) of the full-length or fragmented polypeptide are retained. The term also encompasses the coding region of a structural gene and the sequences located adjacent to the coding region on both the 5′ and 3′ ends for a distance of about 1 kb on either end such that the gene corresponds to the length of the full-length m NA. The sequences that are located 5′ of the coding region and which are present on the mRNA are referred to as “5′ untranslated sequences.” The sequences that are located 3′ (i.e., “downstream”) of the coding region and that are present on the mRNA are referred to as “3′ untranslated sequences.”

The term “gene” encompasses both cDNA and genomic forms of a gene. A genomic form of a genetic clone contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, “based upon” includes assessing, determining, or measuring the individual's characteristics as described herein (and preferably selecting an individual suitable for receiving treatment).

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

The term “about X-Y” used herein has the same meaning as “about X to about Y.” The expression “about X, Y or Z” used herein has the same meaning as “about X, about Y, or about Z.”

As used herein, reference to “not” a value or parameter generally means and describes “other than” a value or parameter. For example, the method is not used to treat cancer of type X means the method is used to treat cancer of types other than X.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

### Methods of Treatment

The present application provides methods of treating graft versus host disease (GvHD) in an individual comprising administering to the individual: an effective amount of an agent comprising IL-22 (such as an IL-22 dimer comprising two monomeric subunits, wherein each monomeric subunit comprises an IL-22 domain and an Fc domain). In some embodiments, the method further comprises administering an immunosuppressive therapy (such as a corticosteroid, which is optionally contraindicated for the individual). In some embodiments, the agent comprising IL-22 (such as the IL-22 dimer) is administered for at least two cycles, wherein each of the at least two cycles comprises administering the IL-22 dimer for a period of at least two weeks, wherein two consecutive cycles of the at least two cycles are separated by a rest period of at least about one week. In some embodiments, the individual has an Ann Arbor score of 3 prior to the treatment. In some embodiments, the individual has an Ann Arbor score of 2 or 1 prior to the treatment. In some embodiments, prior to the treatment, the fecal microbiota of the individual is characterized with a PC2 score of no more than 0.5 based upon analysis of bacterial 16S rRNA sequencing of the fecal sample; or the individual has a gut microbiota profile characterized with enriched Lachnospiraceae and Ruminococcaceae. In some embodiments, the individual has an infection.

In some embodiments, there is provided a method of treating graft versus host disease (GvHD, such as acute GvHD, such as lower GI aGvHD) in an individual, comprising administering to the individual: an effective amount of an agent comprising IL-22 (such as an IL-22 dimer comprising two monomeric subunits, wherein each monomeric subunit comprises an IL-22 domain and an Fc domain, wherein the IL-22 dimer is administered for at least two cycles, wherein each of the at least two cycles comprises administering the IL-22 dimer for a period of at least two weeks, wherein two consecutive cycles of the at least two cycles are separated by a rest period of at least about one week. In some embodiments, there is provided a method of treating graft versus host disease (GvHD, such as acute GvHD, such as lower GI aGvHD) in an individual, comprising administering to the individual: a) an effective amount of an agent comprising IL-22 (such as an IL-22 dimer comprising two monomeric subunits, wherein each monomeric subunit comprises an IL-22 domain and an Fc domain); and b) an immunosuppressive therapy, wherein the IL-22 dimer is administered for at least two cycles, wherein each of the at least two cycles comprises administering the IL-22 dimer for a period of at least two weeks, wherein two consecutive cycles of the at least two cycles are separated by a rest period of at least about one week. In some embodiments, the IL-22 dimer is administered at a frequency of at least about once a week during each of the at least two cycles. In some embodiments, at least one of the at least two cycles comprises administering the IL-22 dimer at least about once a week for at least about four weeks. In some embodiments, each of the at least two cycles comprises administering the IL-22 dimer at least once a week for at least about four weeks. In some embodiments, at least one of the at least two cycles comprises administering the IL-22 dimer no more than about three months. In some embodiments, each of the at least two cycles comprises administering the IL-22 dimer no more than about three months. In some embodiments, the rest period is about one to four weeks. In some embodiments, the rest period is at least about four weeks. In some embodiments, the rest period is no more than three months. In some embodiments, the individual has an Ann Arbor score of 3. In some embodiments, the individual has an Ann Arbor score of 2. In some embodiments, the individual has an Ann Arbor score of 1. In some embodiments, the fecal microbiota of the individual prior to the treatment is characterized with a PC2 score of no more than 0.5 (such as no more than 0) based upon analysis of bacterial 16S rRNA sequencing of the fecal sample. In some embodiments, the individual has a gut microbiota profile characterized with enriched Lachnospiraceae and/or Ruminococcaceae. In some embodiments, the immunosuppressive therapy comprises a corticosteroid, which is optionally contraindicated for the individual. In some embodiments, the IL-22 domain comprises a recombinant IL-22. In some embodiments, said recombinant IL-22 is a human IL-22. In some embodiments, the IL-22 dimer comprises the amino acid sequence of any one of SEQ ID NOS: 6-9. In some embodiments, the IL-22 dimer comprises the amino acid sequence of SEQ ID NO: 6. In some embodiment, the amount of the IL-22 dimer for each administration is about 1 μg/kg to about 500 μg/kg for a human being or a comparable dose for an individual who is not a human being. In some embodiments, the amount of the IL-22 dimer for each administration is about 45 μg/kg for a human being or a comparable dose for an individual who is not a human being. In some embodiments, the IL-22 dimer is administered intravenously.

In some embodiments, there is provided a method of treating graft versus host disease (GvHD, such as acute GvHD, such as lower GI aGvHD) in an individual, comprising administering to the individual: an effective amount of an agent comprising IL-22 (such as an IL-22 dimer comprising two monomeric subunits, wherein each monomeric subunit comprises an IL-22 domain and an Fc domain), wherein the individual has an Ann Arbor score of 3. In some embodiments, there is provided a method of treating graft versus host disease (GvHD, such as acute GvHD, such as lower GI aGvHD) in an individual, comprising administering to the individual: a) an effective amount of an agent comprising IL-22 (such as an IL-22 dimer comprising two monomeric subunits, wherein each monomeric subunit comprises an IL-22 domain and an Fc domain); and b) an immunosuppressive therapy, wherein the individual has an Ann Arbor score of 3. In some embodiments, the fecal microbiota of the individual prior to the treatment is characterized with a PC2 score of no more than 0.5 (such as no more than 0) based upon analysis of bacterial 16S rRNA sequencing of the fecal sample. In some embodiments, the individual has a gut microbiota profile characterized with enriched Lachnospiraceae and/or Ruminococcaceae. In some embodiments, the method further comprises assessing Ann Arbor score of the individual. In some embodiments, the method further comprises selecting the individual for the treatment based upon the individual having an Ann Arbor score of 3. In some embodiments, the immunosuppressive therapy comprises a corticosteroid, which is optionally contraindicated for the individual. In some embodiments, the IL-22 domain comprises a recombinant IL-22. In some embodiments, said recombinant IL-22 is a human IL-22. In some embodiments, the IL-22 dimer comprises the amino acid sequence of any one of SEQ ID NOS: 6-9. In some embodiments, the IL-22 dimer comprises the amino acid sequence of SEQ ID NO: 6. In some embodiment, the amount of the IL-22 dimer for each administration is about 1 μg/kg to about 500 μg/kg for a human being or a comparable dose for an individual who is not a human being. In some embodiments, the amount of the IL-22 dimer for each administration is about 45 μg/kg for a human being or a comparable dose for an individual who is not a human being. In some embodiments, the IL-22 dimer is administered intravenously. In some embodiments, the IL-22 dimer is administered for at least two cycles, wherein each of the at least two cycles comprises administering the IL-22 dimer for a period of at least two weeks, wherein two consecutive cycles of the at least two cycles are separated by a rest period of at least about one week. In some embodiments, the IL-22 dimer is administered at a frequency of at least about once a week during each of the at least two cycles. In some embodiments, at least one of the at least two cycles comprises administering the IL-22 dimer at least about once a week for at least about four weeks. In some embodiments, each of the at least two cycles comprises administering the IL-22 dimer at least once a week for at least about four weeks. In some embodiments, at least one of the at least two cycles comprises administering the IL-22 dimer no more than about three months. In some embodiments, each of the at least two cycles comprises administering the IL-22 dimer no more than about three months. In some embodiments, the rest period is about one to four weeks. In some embodiments, the rest period is at least about four weeks. In some embodiments, the rest period is no more than three months.

In some embodiments, there is provided a method of treating graft versus host disease (GvHD, such as acute GvHD, such as lower GI aGvHD) in an individual, comprising administering to the individual: an effective amount of an agent comprising IL-22 (such as an IL-22 dimer comprising two monomeric subunits, wherein each monomeric subunit comprises an IL-22 domain and an Fc domain), wherein the individual has an Ann Arbor score of 2. In some embodiments, there is provided a method of treating graft versus host disease (GvHD, such as acute GvHD, such as lower GI aGvHD) in an individual, comprising administering to the individual: a) an effective amount of an agent comprising IL-22 (such as an IL-22 dimer comprising two monomeric subunits, wherein each monomeric subunit comprises an IL-22 domain and an Fc domain); and b) an immunosuppressive therapy, wherein the individual has an Ann Arbor score of 2. In some embodiments, the fecal microbiota of the individual prior to the treatment is characterized with a PC2 score of no more than 0.5 (such as no more than 0) based upon analysis of bacterial 16S rRNA sequencing of the fecal sample. In some embodiments, the individual has a gut microbiota profile characterized with enriched Lachnospiraceae and/or Ruminococcaceae. In some embodiments, the method further comprises assessing Ann Arbor score of the individual. In some embodiments, the method further comprises selecting the individual for the treatment based upon the individual having an Ann Arbor score of 2. In some embodiments, the immunosuppressive therapy comprises a corticosteroid, which is optionally contraindicated for the individual. In some embodiments, the IL-22 domain comprises a recombinant IL-22. In some embodiments, said recombinant IL-22 is a human IL-22. In some embodiments, the IL-22 dimer comprises the amino acid sequence of any one of SEQ ID NOS: 6-9. In some embodiments, the IL-22 dimer comprises the amino acid sequence of SEQ ID NO: 6. In some embodiment, the amount of the IL-22 dimer for each administration is about 1 μg/kg to about 500 μg/kg for a human being or a comparable dose for an individual who is not a human being. In some embodiments, the amount of the IL-22 dimer for each administration is about 45 μg/kg for a human being or a comparable dose for an individual who is not a human being. In some embodiments, the IL-22 dimer is administered intravenously. In some embodiments, the IL-22 dimer is administered for at least two cycles, wherein each of the at least two cycles comprises administering the IL-22 dimer for a period of at least two weeks, wherein two consecutive cycles of the at least two cycles are separated by a rest period of at least about one week. In some embodiments, the IL-22 dimer is administered at a frequency of at least about once a week during each of the at least two cycles. In some embodiments, at least one of the at least two cycles comprises administering the IL-22 dimer at least about once a week for at least about four weeks. In some embodiments, each of the at least two cycles comprises administering the IL-22 dimer at least once a week for at least about four weeks. In some embodiments, at least one of the at least two cycles comprises administering the IL-22 dimer no more than about three months. In some embodiments, each of the at least two cycles comprises administering the IL-22 dimer no more than about three months. In some embodiments, the rest period is about one to four weeks. In some embodiments, the rest period is at least about four weeks. In some embodiments, the rest period is no more than three months.

In some embodiments, there is provided a method of treating graft versus host disease (GvHD, such as acute GvHD, such as lower GI aGvHD) in an individual, comprising administering to the individual: an effective amount of an agent comprising IL-22 (such as an IL-22 dimer comprising two monomeric subunits, wherein each monomeric subunit comprises an IL-22 domain and an Fc domain), wherein the individual has an Ann Arbor score of 1. In some embodiments, there is provided a method of treating graft versus host disease (GvHD, such as acute GvHD, such as lower GI aGvHD) in an individual, comprising administering to the individual: a) an effective amount of an agent comprising IL-22 (such as an IL-22 dimer comprising two monomeric subunits, wherein each monomeric subunit comprises an IL-22 domain and an Fc domain); and b) an immunosuppressive therapy, wherein the individual has an Ann Arbor score of 1. In some embodiments, the fecal microbiota of the individual prior to the treatment is characterized with a PC2 score of no more than 0.5 (such as no more than 0) based upon analysis of bacterial 16S rRNA sequencing of the fecal sample. In some embodiments, the individual has a gut microbiota profile characterized with enriched Lachnospiraceae and/or Ruminococcaceae. In some embodiments, the method further comprises assessing Ann Arbor score of the individual. In some embodiments, the method further comprises selecting the individual for the treatment based upon the individual having an Ann Arbor score of 1. In some embodiments, the immunosuppressive therapy comprises a corticosteroid, which is optionally contraindicated for the individual. In some embodiments, the IL-22 domain comprises a recombinant IL-22. In some embodiments, said recombinant IL-22 is a human IL-22. In some embodiments, the IL-22 dimer comprises the amino acid sequence of any one of SEQ ID NOS: 6-9. In some embodiments, the IL-22 dimer comprises the amino acid sequence of SEQ ID NO: 6. In some embodiment, the amount of the IL-22 dimer for each administration is about 1 μg/kg to about 500 μg/kg for a human being or a comparable dose for an individual who is not a human being. In some embodiments, the amount of the IL-22 dimer for each administration is about 45 μg/kg for a human being or a comparable dose for an individual who is not a human being. In some embodiments, the IL-22 dimer is administered intravenously. In some embodiments, the IL-22 dimer is administered for at least two cycles, wherein each of the at least two cycles comprises administering the IL-22 dimer for a period of at least two weeks, wherein two consecutive cycles of the at least two cycles are separated by a rest period of at least about one week. In some embodiments, the IL-22 dimer is administered at a frequency of at least about once a week during each of the at least two cycles. In some embodiments, at least one of the at least two cycles comprises administering the IL-22 dimer at least about once a week for at least about four weeks. In some embodiments, each of the at least two cycles comprises administering the IL-22 dimer at least once a week for at least about four weeks. In some embodiments, at least one of the at least two cycles comprises administering the IL-22 dimer no more than about three months. In some embodiments, each of the at least two cycles comprises administering the IL-22 dimer no more than about three months. In some embodiments, the rest period is about one to four weeks. In some embodiments, the rest period is at least about four weeks. In some embodiments, the rest period is no more than three months.

In some embodiments, there is provided a method of treating graft versus host disease (GvHD, such as acute GvHD, such as lower GI aGvHD) in an individual, comprising administering to the individual: an effective amount of an agent comprising IL-22 (such as an IL-22 dimer comprising two monomeric subunits, wherein each monomeric subunit comprises an IL-22 domain and an Fc domain), wherein prior to the treatment: i) the fecal microbiota of the individual is characterized with a PC2 score of no more than 0.5 based upon analysis of bacterial 16S rRNA sequencing of the fecal sample; or ii) the individual has a gut microbiota profile characterized with enriched Lachnospiraceae and Ruminococcaceae. In some embodiments, the method further comprises administering to the individual an immunosuppressive therapy. In some embodiments, the method further comprises assessing bacterial 16s rRNA gene sequence in fecal sample of the individual. In some embodiments, the method further comprises selecting the individual for the treatment based upon the fecal microbiota of the individual having a PC2 score of no more than 0.5 based upon analysis of bacterial 16S rRNA sequencing of the fecal sample. In some embodiments, the immunosuppressive therapy comprises a corticosteroid, which is optionally contraindicated for the individual. In some embodiments, the IL-22 domain comprises a recombinant IL-22. In some embodiments, said recombinant IL-22 is a human IL-22. In some embodiments, the IL-22 dimer comprises the amino acid sequence of any one of SEQ ID NOS: 6-9. In some embodiments, the IL-22 dimer comprises the amino acid sequence of SEQ ID NO: 6. In some embodiment, the amount of the IL-22 dimer for each administration is about 1 μg/kg to about 500 μg/kg for a human being or a comparable dose for an individual who is not a human being. In some embodiments, the amount of the IL-22 dimer for each administration is about 45 μg/kg for a human being or a comparable dose for an individual who is not a human being. In some embodiments, the IL-22 dimer is administered intravenously. In some embodiments, the IL-22 dimer is administered for at least two cycles, wherein each of the at least two cycles comprises administering the IL-22 dimer for a period of at least two weeks, wherein two consecutive cycles of the at least two cycles are separated by a rest period of at least about one week. In some embodiments, the IL-22 dimer is administered at a frequency of at least about once a week during each of the at least two cycles. In some embodiments, at least one of the at least two cycles comprises administering the IL-22 dimer at least about once a week for at least about four weeks. In some embodiments, each of the at least two cycles comprises administering the IL-22 dimer at least once a week for at least about four weeks. In some embodiments, at least one of the at least two cycles comprises administering the IL-22 dimer no more than about three months. In some embodiments, each of the at least two cycles comprises administering the IL-22 dimer no more than about three months. In some embodiments, the rest period is about one to four weeks. In some embodiments, the rest period is at least about four weeks. In some embodiments, the rest period is no more than three months.

In some embodiments, there is provided a method of treating graft versus host disease (GvHD, such as acute GvHD, such as lower GI aGvHD) in an individual, comprising administering to the individual: a) an effective amount of an agent comprising IL-22 (such as an IL-22 dimer comprising two monomeric subunits, wherein each monomeric subunit comprises an IL-22 domain and an Fc domain), b) an immunosuppressive therapy, wherein the individual has an infection. In some embodiments, the immunosuppressive therapy comprises a corticosteroid, which is optionally contraindicated for the individual. In some embodiments, the IL-22 domain comprises a recombinant IL-22. In some embodiments, said recombinant IL-22 is a human IL-22. In some embodiments, the IL-22 dimer comprises the amino acid sequence of any one of SEQ ID NOS: 6-9. In some embodiments, the IL-22 dimer comprises the amino acid sequence of SEQ ID NO: 6. In some embodiment, the amount of the IL-22 dimer for each administration is about 1 μg/kg to about 500 μg/kg for a human being or a comparable dose for an individual who is not a human being. In some embodiments, the amount of the IL-22 dimer for each administration is about 45 μg/kg for a human being or a comparable dose for an individual who is not a human being. In some embodiments, the IL-22 dimer is administered intravenously. In some embodiments, the IL-22 dimer is administered for at least two cycles, wherein each of the at least two cycles comprises administering the IL-22 dimer for a period of at least two weeks, wherein two consecutive cycles of the at least two cycles are separated by a rest period of at least about one week. In some embodiments, the IL-22 dimer is administered at a frequency of at least about once a week during each of the at least two cycles. In some embodiments, at least one of the at least two cycles comprises administering the IL-22 dimer at least about once a week for at least about four weeks. In some embodiments, each of the at least two cycles comprises administering the IL-22 dimer at least once a week for at least about four weeks. In some embodiments, at least one of the at least two cycles comprises administering the IL-22 dimer no more than about three months. In some embodiments, each of the at least two cycles comprises administering the IL-22 dimer no more than about three months. In some embodiments, the rest period is about one to four weeks. In some embodiments, the rest period is at least about four weeks. In some embodiments, the rest period is no more than three months.

In some embodiments, there is provided a method of treating graft versus host disease (GvHD, such as acute GvHD, such as lower GI aGvHD) in an individual, comprising administering to the individual: a) an effective amount of an agent comprising IL-22 (such as an IL-22 dimer comprising two monomeric subunits, wherein each monomeric subunit comprises an IL-22 domain and an Fc domain), b) an immunosuppressive therapy, wherein the IL-22 dimer is administered for at least two cycles, wherein each of the at least two cycles comprises administering the IL-22 dimer for a period of at least two weeks, wherein two consecutive cycles of the at least two cycles are separated by a rest period of at least about one week, and wherein the individual has an Ann Arbor score of 3 prior to the treatment. In some embodiments, at least one of the at least two cycles comprises administering the IL-22 dimer at least about once a week for at least about four weeks. In some embodiments, each of the at least two cycles comprises administering the IL-22 dimer at least once a week for at least about four weeks. In some embodiments, at least one of the at least two cycles comprises administering the IL-22 dimer no more than about three months. In some embodiments, each of the at least two cycles comprises administering the IL-22 dimer no more than about three months. In some embodiments, the rest period is about one to four weeks. In some embodiments, the rest period is at least about four weeks. In some embodiments, the rest period is no more than three months. In some embodiments, the immunosuppressive therapy comprises a corticosteroid, which is optionally contraindicated for the individual. In some embodiments, the IL-22 domain comprises a recombinant IL-22. In some embodiments, said recombinant IL-22 is a human IL-22. In some embodiments, the IL-22 dimer comprises the amino acid sequence of any one of SEQ ID NOS: 6-9. In some embodiments, the IL-22 dimer comprises the amino acid sequence of SEQ ID NO: 6. In some embodiment, the amount of the IL-22 dimer for each administration is about 1 μg/kg to about 500 μg/kg for a human being or a comparable dose for an individual who is not a human being. In some embodiments, the amount of the IL-22 dimer for each administration is about 45 μg/kg for a human being or a comparable dose for an individual who is not a human being. In some embodiments, the IL-22 dimer is administered intravenously.

In some embodiments, there is provided a method of treating graft versus host disease (GvHD, such as acute GvHD, such as lower GI aGvHD) in an individual, comprising administering to the individual: a) an effective amount of an agent comprising IL-22 (such as an IL-22 dimer comprising two monomeric subunits, wherein each monomeric subunit comprises an IL-22 domain and an Fc domain), b) an immunosuppressive therapy, wherein the IL-22 dimer is administered for at least two cycles, wherein each of the at least two cycles comprises administering the IL-22 dimer for a period of at least two weeks, wherein two consecutive cycles of the at least two cycles are separated by a rest period of at least about one week, and wherein the individual has an Ann Arbor score of 2 prior to the treatment. In some embodiments, at least one of the at least two cycles comprises administering the IL-22 dimer at least about once a week for at least about four weeks. In some embodiments, each of the at least two cycles comprises administering the IL-22 dimer at least once a week for at least about four weeks. In some embodiments, at least one of the at least two cycles comprises administering the IL-22 dimer no more than about three months. In some embodiments, each of the at least two cycles comprises administering the IL-22 dimer no more than about three months. In some embodiments, the rest period is about one to four weeks. In some embodiments, the rest period is at least about four weeks. In some embodiments, the rest period is no more than three months. In some embodiments, the immunosuppressive therapy comprises a corticosteroid, which is optionally contraindicated for the individual. In some embodiments, the IL-22 domain comprises a recombinant IL-22. In some embodiments, said recombinant IL-22 is a human IL-22. In some embodiments, the IL-22 dimer comprises the amino acid sequence of any one of SEQ ID NOS: 6-9. In some embodiments, the IL-22 dimer comprises the amino acid sequence of SEQ ID NO: 6. In some embodiment, the amount of the IL-22 dimer for each administration is about 1 μg/kg to about 500 μg/kg for a human being or a comparable dose for an individual who is not a human being. In some embodiments, the amount of the IL-22 dimer for each administration is about 45 μg/kg for a human being or a comparable dose for an individual who is not a human being. In some embodiments, the IL-22 dimer is administered intravenously.

In some embodiments, there is provided a method of treating graft versus host disease (GvHD, such as acute GvHD, such as lower GI aGvHD) in an individual, comprising administering to the individual: a) an effective amount of an agent comprising IL-22 (such as an IL-22 dimer comprising two monomeric subunits, wherein each monomeric subunit comprises an IL-22 domain and an Fc domain), b) an immunosuppressive therapy, wherein the IL-22 dimer is administered for at least two cycles, wherein each of the at least two cycles comprises administering the IL-22 dimer for a period of at least two weeks, wherein two consecutive cycles of the at least two cycles are separated by a rest period of at least about one week, and wherein the individual has an Ann Arbor score of 1 prior to the treatment. In some embodiments, at least one of the at least two cycles comprises administering the IL-22 dimer at least about once a week for at least about four weeks. In some embodiments, each of the at least two cycles comprises administering the IL-22 dimer at least once a week for at least about four weeks. In some embodiments, at least one of the at least two cycles comprises administering the IL-22 dimer no more than about three months. In some embodiments, each of the at least two cycles comprises administering the IL-22 dimer no more than about three months. In some embodiments, the rest period is about one to four weeks. In some embodiments, the rest period is at least about four weeks. In some embodiments, the rest period is no more than three months. In some embodiments, the immunosuppressive therapy comprises a corticosteroid, which is optionally contraindicated for the individual. In some embodiments, the IL-22 domain comprises a recombinant IL-22. In some embodiments, said recombinant IL-22 is a human IL-22. In some embodiments, the IL-22 dimer comprises the amino acid sequence of any one of SEQ ID NOS: 6-9. In some embodiments, the IL-22 dimer comprises the amino acid sequence of SEQ ID NO: 6. In some embodiment, the amount of the IL-22 dimer for each administration is about 1 μg/kg to about 500 μg/kg for a human being or a comparable dose for an individual who is not a human being. In some embodiments, the amount of the IL-22 dimer for each administration is about 45 μg/kg for a human being or a comparable dose for an individual who is not a human being. In some embodiments, the IL-22 dimer is administered intravenously.

In some embodiments, there is provided a method of treating graft versus host disease (GvHD, such as acute GvHD, such as lower GI aGvHD) in an individual, comprising administering to the individual: a) an effective amount of an agent comprising IL-22 (such as an IL-22 dimer comprising two monomeric subunits, wherein each monomeric subunit comprises an IL-22 domain and an Fc domain), b) an immunosuppressive therapy, wherein the IL-22 dimer is administered for at least two cycles, wherein each of the at least two cycles comprises administering the IL-22 dimer for a period of at least two weeks, wherein two consecutive cycles of the at least two cycles are separated by a rest period of at least about one week, and wherein prior to the treatment: i) the fecal microbiota of the individual is characterized with a PC2 score of no more than 0.5 based upon analysis of bacterial 16S rRNA sequencing of the fecal sample; or ii) the individual has a gut microbiota profile characterized with enriched Lachnospiraceae and Ruminococcaceae. In some embodiments, at least one of the at least two cycles comprises administering the IL-22 dimer at least about once a week for at least about four weeks. In some embodiments, each of the at least two cycles comprises administering the IL-22 dimer at least once a week for at least about four weeks. In some embodiments, at least one of the at least two cycles comprises administering the IL-22 dimer no more than about three months. In some embodiments, each of the at least two cycles comprises administering the IL-22 dimer no more than about three months. In some embodiments, the rest period is about one to four weeks. In some embodiments, the rest period is at least about four weeks. In some embodiments, the rest period is no more than three months. In some embodiments, the immunosuppressive therapy comprises a corticosteroid, which is optionally contraindicated for the individual. In some embodiments, the IL-22 domain comprises a recombinant IL-22. In some embodiments, said recombinant IL-22 is a human IL-22. In some embodiments, the IL-22 dimer comprises the amino acid sequence of any one of SEQ ID NOS: 6-9. In some embodiments, the IL-22 dimer comprises the amino acid sequence of SEQ ID NO: 6. In some embodiment, the amount of the IL-22 dimer for each administration is about 1 μg/kg to about 500 μg/kg for a human being or a comparable dose for an individual who is not a human being. In some embodiments, the amount of the IL-22 dimer for each administration is about 45 μg/kg for a human being or a comparable dose for an individual who is not a human being. In some embodiments, the IL-22 dimer is administered intravenously.

In some embodiments, there is provided a method of treating graft versus host disease (GvHD, such as acute GvHD, such as lower GI aGvHD) in an individual, comprising administering to the individual: a) an effective amount of an agent comprising IL-22 (such as an IL-22 dimer comprising two monomeric subunits, wherein each monomeric subunit comprises an IL-22 domain and an Fc domain), b) an immunosuppressive therapy, wherein the IL-22 dimer is administered for at least two cycles, wherein each of the at least two cycles comprises administering the IL-22 dimer for a period of at least two weeks, wherein two consecutive cycles of the at least two cycles are separated by a rest period of at least about one week, and wherein the individual has an infection.

In some embodiments, the GvHD is acute GvHD. In some embodiments, the GvHD is a gastrointestinal GvHD (GI GvHD). In some embodiments, the GI GvHD is lower GI GvHD. In some embodiments, the lower GI GvHD is selected from the group consisting of Grade II, Grade III and Grade IV lower GI GvHD. In some embodiments, the individual has not been subject to corticosteroid for GvHD for a period of three or more days prior to the treatment. In some embodiments, the individual does not have an ongoing Cytomegalovirus (CMV) infection immediately prior to the treatment. In some embodiments, the individual is a human. In some embodiments, the immunosuppressive therapy comprises a corticosteroid drug. In some embodiments, the corticosteroid drug is a systemic corticosteroid. In some embodiments, the corticosteroid drug is a prednisone. In some embodiments, the prednisone is administered at a dose of no more than about 2 mg/kg for a human being or a comparable dose for an individual who is not a human being. In some embodiments, the corticosteroid drug is administered daily.

In some embodiments, there is provided a method of treating graft versus host disease (GvHD, such as acute GvHD, such as lower GI aGvHD) in an individual, comprising administering to the individual: a) an effective amount of an IL-22 dimer comprising two monomeric subunits, wherein each monomeric subunit comprises an IL-22 domain and an Fc domain, wherein the IL-22 dimer comprises the amino acid sequence of any one of SEQ ID NOS: 6-9; and b) a corticosteroid drug (e.g., a systemic corticosteroid, e.g., prednisone), wherein the IL-22 dimer is administered for at least two cycles, wherein each of the at least two cycles comprises administering the IL-22 dimer for a period of at least two weeks, wherein two consecutive cycles of the at least two cycles are separated by a rest period of at least about one week.

In some embodiments, there is provided a method of treating graft versus host disease (GvHD, such as acute GvHD, such as lower GI aGvHD) in an individual, comprising administering to the individual: a) an effective amount of an IL-22 dimer comprising two monomeric subunits, wherein each monomeric subunit comprises an IL-22 domain and an Fc domain, wherein the IL-22 dimer comprises the amino acid sequence of any one of SEQ ID NOS: 6-9; and b) a corticosteroid drug (e.g., a systemic corticosteroid, e.g., prednisone), and wherein prior to the treatment: i) the fecal microbiota of the individual is characterized with a PC2 score of no more than 0.5 based upon analysis of bacterial 16S rRNA sequencing of the fecal sample; or ii) the individual has a gut microbiota profile characterized with enriched Lachnospiraceae and Ruminococcaceae.

In some embodiments, there is provided a method of treating graft versus host disease (GvHD, such as acute GvHD, such as lower GI aGvHD) in an individual, comprising administering to the individual: a) an effective amount of an IL-22 dimer comprising two monomeric subunits, wherein each monomeric subunit comprises an IL-22 domain and an Fc domain, wherein the IL-22 dimer comprises the amino acid sequence of any one of SEQ ID NOS: 6-9; and b) a corticosteroid drug (e.g., a systemic corticosteroid, e.g., prednisone), wherein the individual has an infection.

In some embodiments, the method further comprises administering a second or third agent or therapy. In some embodiments, the second or third agent or therapy is a standard or commonly used agent or therapy for treating GvHD. In some embodiments, the second or third agent or therapy modulates gut microbiota. For example, in some embodiments, the second or third agent or therapy promotes a more abundant and/or diverse gut microbiota profile. In some embodiments, the second or third agent or therapy promotes a gut microbiota profile characterized with enriched Lachnospiraceae and/or Ruminococcaceae in gut microbiota in the individual. In some embodiments, the agent (such as an IL-22 dimer) and the second/third agent or therapy are administered sequentially. In some embodiments, the agent (such as an IL-22 dimer) and the second/third agent or therapy are administered concurrently. In some embodiments, the agent (such as an IL-22 dimer) and the second/third agent or therapy are administered simultaneously.

### Agent Comprising IL-22

The agents that comprise an IL-22 domain described herein include, and not limited to, an IL-22 or variant thereof and various IL-22 fusion proteins, such as an IL-22 dimer, such as an IL-22 dimer.

In some embodiments, the agent comprising IL-22 is an IL-22 fusion protein comprising a) an IL-22 domain, and b) a half-life prolonging domain. In some embodiments, the IL-22 domain comprises a recombinant IL-22. In some embodiments, the recombinant IL-22 is a human IL-22. In some embodiments, the half-life prolonging domain comprises an Fc domain (such as any of the Fc domain described herein). In some embodiments, the half-life prolonging domain comprises an albumin-binding domain (such as an anti-albumin domain).

### IL-22

Interleukin-22 (IL-22) is protein that in humans is encoded by the IL22 gene (also known as TIFα, IL-21, ILTIF, IL-TIF, IL-D110, zcyto18, TIFIL-23). Native IL-22 precursor peptide consists of 179 amino acid residues, while the mature peptide consists of 146 amino acid residues.

IL-22 is an α-helical cytokine. IL-22 binds to a heterodimeric cell surface receptor composed of IL-10R2 and IL-22R1 subunits. IL-22R is expressed on tissue cells, and it is absent on immune cells. IL-22 is produced by several populations of immune cells at a site of inflammation. Producers include αβ T cells classes Th1, Th22 and Th17 along with γδ T cells, NKT, ILC3, neutrophils and macrophages. IL-22 takes effect on non-hematopoietic cells—mainly stromal and epithelial cells. Effects involve stimulation of cell survival, proliferation and synthesis of antimicrobials including S100, Reg3β, Reg3γ and defensins. IL-22 thus participates in both wound healing and in protection against microbes. IL-22 dysregulation takes part in pathogenesis of several autoimmune diseases like systemic lupus erythematosus, rheumatoid arthritis and psoriasis.

The use of IL-22 was reported for use in the treatment of human diseases (for example, U.S. Pat. No. 6,551,799, U.S. patent application No. 20130171100, PCT patent application No. WO2015070077, herein incorporated by reference in their entirety).

IL-22 has been shown to support intestinal mucosa after damage through multiple mechanisms, including direct signaling to the intestinal epithelium that promotes its survival and regeneration as well by inducing epithelial production of innate antimicrobial antimicrobial molecules such as REG3. In experimental murine models, aGvHD resulted in elimination of host-derived IL-22-producing cells, IL-22 deficiency increased GvHD mortality and GI pathology, and early initiation of treatment with exogenous IL-22 reduced GI pathology and improved survival.

An agent comprising an IL-22 domain for use in practicing the method of the present invention can not only be generated using recombinant DNA technology, but can also be produced through fusion of heterologous polypeptides. A description of other methods, vectors and host cells for synthesis of an IL-22 for use in practicing the method of the present invention can be found in Gething et al, Nature, 293:620-625; Mantei et al, Nature, 281:40-46. Recombinant IL-22, dimers and fusion proteins thereof are produced using methods known to those of skill in the art. For more details, see U.S. published application no. 2013/0171100, the contents of which are hereby incorporated by reference into the present disclosure.

DNA sequences encoding an IL-22 dimer or fusion protein can be entirely synthesized artificially. Alternatively, DNA encoding IL-22 can be obtained by PCR amplification or synthesis and then joined together to form a DNA sequence encoding an IL-22 dimer.

Briefly, suitable host cells are transformed or transfected with an IL-22 expression vector in accordance with known methods and subsequently grown under conditions known to those of skill in the art to promote growth; transfection techniques are described in greater detail in, for example, Molecular Cloning: a Laboratory Manual 3rd edition, J. F. Sambrook and D. W. Russell, ed. Cold Spring Harbor Laboratory Press 2001.

Host cells suitable for expression of IL-22 are also known in the art and include invertebrate cells, such as insect cells, and mammalian cells. Suitable mammalian cells include Chinese Hamster Ovary (CHO), COS cells; in particular, SV40-transformed monkey kidney CV1 cell line (COS-7, ATCC CRL 1651); human embryo kidney cell line 293 (Graham et al, J. Gen Virol, 36:59 (1997)); CHO/−DHFR (Urlaub and Chasin, Proc. Natl. Acad. Sci. USA, 77:4216 (1980)); murine testis trophoblastic cells (TM4, Mather, Biol. Reprod., 23:243-251) (1980)); human lung cells (W138, ATCC CCL 75); human liver cells (Hep G2, HB 8065); murine breast cancer cells (MMT 060562, ATCC CCL51).

A nucleic acid comprising a nucleotide sequence encoding IL-22 can be inserted into a replicable vector for gene cloning or protein expression. Many vectors for protein expression are known in the art. Using these techniques, a nucleic acid sequence encoding IL-22 is inserted into an appropriate vector, which may further include any of the following: one or more signal sequences, an origin of replication, one or more reporter genes, an enhancer element, a promoter, and a transcription termination sequence.

Methods of transfecting eukaryotic cells and transforming prokaryotic cells are also known to those of skill in the art, and may include the use of calcium chloride, calcium phosphate precipitation, lipofectamine or electroporation. One skilled in the art will be able to select a suitable method depending on the host cell selected.

In some embodiments, the agent comprising an IL-22 domain is an IL-22. In some embodiments, the IL-22 is a recombinant IL-22 (rIL-22). In some embodiments, the IL-22 is a human IL-22. In some embodiments, the IL-22 is a recombinant human IL-22.

In some embodiments, the IL-22 is an IL-22 monomer. In some embodiments, the IL-22 monomer comprises the amino acid sequence of SEQ ID NO: 1.

In some embodiments, the agent comprising an IL-22 domain is an IL-22 fusion protein comprising a) an IL-22 domain, and b) a half-life extending domain. In some embodiments, the half-life extending domain is an Fc domain, such as any of the Fc domains described herein. In some embodiments, the half-life extending domain is an albumin binding domain (such as an anti-albumin antibody).

### IL-22 Dimer

In some embodiments, the IL-22 is an IL-22 dimer. In some embodiments, the IL-22 dimer has exactly two units of IL-22. In some embodiments, one or both units of IL-22 comprise the amino acid sequence of SEQ ID NO: 1.

In some embodiments, the IL-22 dimer comprises two monomeric subunits, wherein each monomeric subunit comprises an IL-22 domain and a dimerization domain. In some embodiments, the IL-22 domain is fused to the N-terminus of the dimerization domain. In some embodiments, the IL-22 domain is fused to the C-terminus of the dimerization domain. In some embodiments, the IL-22 domain is fused to the dimerization domain via a linker, such as any of the linkers described herein. In some embodiments, the linker is a peptide linker. In some embodiments, the linker has a length of about one to 50 amino acids (such as about three to thirty amino acids, such as about four to twenty-five amino acids, such as about five to twenty amino acids). In some embodiments, the linker comprises or has an amino acid sequence of any one of SEQ ID NOS: 4, 5, and 11-19. In some embodiments, the linker comprises has an amino acid sequence of SEQ ID NO: 4 or 5.

The IL-22 dimer described herein comprises two monomeric subunits, wherein each monomeric subunit comprises an IL-22 domain and a dimerization domain that is an Fc domain, such as any of the Fc domains described herein. In some embodiments, the IL-22 domain is fused to the N-terminus or C-terminus of the Fc domain via a linker. In some embodiments, the linker is a peptide linker. In some embodiments, the linker has a length of about one to 50 amino acids (such as about three to thirty amino acids, such as about four to twenty-five amino acids, such as about five to twenty amino acids). In some embodiments, the linker comprises or has an amino acid sequence of any one of SEQ ID NOS: 4, 5, and 11-19. In some embodiments, the linker comprises has an amino acid sequence of SEQ ID NO: 4 or 5.

In some embodiments, the IL-22 domain comprises a recombinant IL-22. In some embodiments, the recombinant IL-22 is a human IL-22. In some embodiments, the IL-22 domain has an IL-22 monomer. In some embodiments, the IL-22 domain monomer comprises the amino acid sequence of SEQ ID NO: 1.

In some embodiments, the IL-22 dimer comprises an amino acid sequence selected from the group consisting of SEQ ID NOS: 6-9. In some embodiments, the IL-22 dimer comprises an amino acid sequence of SEQ ID NO: 6.

### Fc Domain

The term “Fc domain” herein refers to a domain that comprises a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the molecule that has the Fc region.

Suitable native-sequence Fc regions for use in the agent comprising IL-22 (such as IL-22 dimer) described herein include human IgG1, IgG2 (IgG2A, IgG2B), IgG3 and IgG4.

In some embodiments, the Fc domain comprises the Fc region of human IgG1, IgG2, IgG3, IgG4, or a combination or hybrid IgG. In some embodiments, the Fc domain is an IgG2 Fc region (such as a human IgG2 Fc region). In some embodiments, the Fc domain comprises an amino acid sequence of SEQ ID NO: 2. In some embodiments, the Fc domain is an IgG4 Fc region (such as a human IgG4 Fc region). In some embodiments, the Fc domain comprises an amino acid sequence of SEQ ID NO: 3.

### Fc Region Variants

In some embodiments, one or more amino acid modifications may be introduced into the Fc region, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In some embodiments, the FC domain possesses some but not all effector functions, which make it a desirable candidate for applications in which the half-life of the agent comprising IL-22 (such as IL-22 dimer) in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the agent comprising IL-22 (such as IL-22 dimer) lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 2 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)).

Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the agent comprising IL-22 (such as IL-22 dimer) is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12): 1759-1769 (2006)).

Fc region variants with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain Fc region variants with improved or diminished binding to FcRs are described. (See, e.g., U.S. Pat. No. 6,737,056; WO 2004/056312, and Shields et al., J. Biol. Chem. 9(2): 6591-6604 (2001).)

In some embodiments, the FC domain is an IgG1 Fc region. In some embodiments, the IgG1 Fc fragment comprises a L234A mutation and/or a L235A mutation. In some embodiments, the Fc fragment is an IgG2 or IgG4 Fc fragment. In some embodiments, the Fc fragment is an IgG4 Fc fragment comprising a S228P, F234A, and/or a L235A mutation.

In some embodiments, the Fc domain comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164:4178-4184 (2000).

In some embodiments, the Fc domain comprises a variant Fc region comprising one or more amino acid substitutions which alters half-life and/or changes binding to the neonatal Fc receptor (FcRn). Fc region variants with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those Fc regions have one or more substitutions therein which alters binding of the Fc region to FcRn. Such Fc region variants include those with substitutions at one or more of Fc region residues, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

### Linkers

In some embodiments, the agent comprising an IL-22 domain (e.g., IL-22 dimer) described herein comprise one or more linkers (such as the linker between the IL-22 domain and dimerization domain). The length, the degree of flexibility and/or other properties of the linkers used in the agent comprising an IL-22 domain (e.g., IL-22 dimer) may have some influence on properties of the IL-22 domain or any other domains in the agent. For example, longer linkers may be selected to ensure that two adjacent domains do not sterically interfere with one another. In some embodiment, a linker (such as peptide linker) comprises flexible residues (such as glycine and serine) so that the adjacent domains are free to move relative to each other. For example, a glycine-serine doublet can be a suitable peptide linker. In some embodiments, the linker is a non-peptide linker. In some embodiments, the linker is a peptide linker.

Other linker considerations include the effect on physical or pharmacokinetic properties of the resulting compound, such as solubility, lipophilicity, hydrophilicity, hydrophobicity, stability (more or less stable as well as planned degradation), rigidity, flexibility, immunogenicity, modulation of agent binding, the ability to be incorporated into a micelle or liposome, and the like.

In some embodiments, the linker (or one or more of the linkers) is a peptide linker. In some embodiments, the linker (or one or more of the linkers) is a non-peptide linker.

### Peptide Linkers

The peptide linker may have a naturally occurring sequence, or a non-naturally occurring sequence. For example, a sequence derived from the hinge region of heavy chain only antibodies may be used as the linker. See, for example, WO1996/34103.

The peptide linker can be of any suitable length. In some embodiments, the peptide linker is at least about any of 1, 2, 3, 4, 5, 6, 7, 8, 9, 10, 11, 12, 13, 14, 15, 16, 17, 18, 19, 20, 25, 30, 35, 40, 50, 75, 100 or more amino acids long. In some embodiments, the peptide linker is no more than about any of 100, 75, 50, 40, 35, 30, 25, 20, 19, 18, 17, 16, 15, 14, 13, 12, 11, 10, 9, 8, 7, 6, 5 or fewer amino acids long. In some embodiments, the length of the peptide linker is any of about 1 amino acid to about 10 amino acids, about 1 amino acid to about 20 amino acids, about 1 amino acid to about 30 amino acids, about 5 amino acids to about 15 amino acids, about 10 amino acids to about 25 amino acids, about 5 amino acids to about 30 amino acids, about 10 amino acids to about 30 amino acids long, about 30 amino acids to about 50 amino acids, about 50 amino acids to about 100 amino acids, or about 1 amino acid to about 100 amino acids.

An essential technical feature of such peptide linker is that said peptide linker does not comprise any polymerization activity. The characteristics of a peptide linker, which comprise the absence of the promotion of secondary structures, are known in the art and described, e.g., in Dall'Acqua et al. (Biochem. (1998) 37, 9266-9273), Cheadle et al. (Mol Immunol (1992) 29, 21-30) and Raag and Whitlow (FASEB (1995) 9(1), 73-80). A particularly preferred amino acid in context of the “peptide linker” is Gly. Furthermore, peptide linkers that also do not promote any secondary structures are preferred. The linkage of the domains to each other can be provided by, e.g., genetic engineering. Methods for preparing fused and operatively linked agents such as those described in the present application and expressing them in mammalian cells or bacteria are well-known in the art (e.g. WO 99/54440, Ausubel, Current Protocols in Molecular Biology, Green Publishing Associates and Wiley Interscience, N. Y. 1989 and 1994 or Sambrook et al., Molecular Cloning: A Laboratory Manual, Cold Spring Harbor Laboratory Press, Cold Spring Harbor, N. Y., 2001).

The peptide linker can be a stable linker, which is not cleavable by proteases, especially by Matrix metalloproteinases (MMPs).

The linker can also be a flexible linker. Exemplary flexible linkers include glycine polymers (G)ₙ (SEQ ID NO: 11), glycine-serine polymers (including, for example, (GS)ₙ (SEQ ID NO: 12)ₙ (GSGGS)ₙ (SEQ ID NO: 13)ₙ (GGGGS)ₙ (SEQ ID NO: 14), and (GGGS)ₙ (SEQ ID NO: 15), where n is an integer of at least one), glycine-alanine polymers, alanine-serine polymers, and other flexible linkers known in the art. Glycine and glycine-serine polymers are relatively unstructured, and therefore may be able to serve as a neutral tether between components. Glycine accesses significantly more phi-psi space than even alanine, and is much less restricted than residues with longer side chains (see Scheraga, Rev. Computational Chem. 11 173-142 (1992)). The ordinarily skilled artisan will recognize that design of the agent (such as an IL-22 fusion protein, such as an IL-22 dimer) can include linkers that are all or partially flexible, such that the linker can include a flexible linker portion as well as one or more portions that confer less flexible structure to provide a desired agent structure.

Further, a linker may comprise, for example, the amino acid sequence of such as (GGGGS)ₙ (SEQ ID NO: 16), wherein n is an integer between 1 and 8, e.g. (GGGGS)₃ (SEQ ID NO: 17; hereinafter referred to as “(G4S)3” or “GS3”), or (GGGGS)₆ (SEQ ID NO: 18; hereinafter referred to as “(G4S)6” or “GS6”). In some embodiments, the peptide linker comprises the amino acid sequence of (GSTSGSGKPGSGEGS)ₙ (SEQ ID NO: 19), wherein n is an integer between 1 and 3.

### Non-Peptide Linkers

Coupling of two moieties may be accomplished by any chemical reaction that will bind the two molecules so long as both components retain their respective activities. This linkage can include many chemical mechanisms, for instance covalent binding, affinity binding, intercalation, coordinate binding and complexation. In some embodiments, the binding is covalent binding. Covalent binding can be achieved either by direct condensation of existing side chains or by the incorporation of external bridging molecules. Many bivalent or polyvalent linking agents may be useful in coupling protein molecules in this context. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents (see Killen and Lindstrom, Jour. Immun. 133:1335-2549 (1984); Jansen et al., Immunological Reviews 62:185-216 (1982); and Vitetta et al., Science 238:1098 (1987)).

Linkers that can be applied in the present application are described in the literature (see, for example, Ramakrishnan, S. et al., Cancer Res. 44:201-208 (1984) describing use of MBS (M-maleimidobenzoyl-N-hydroxysuccinimide ester). In some embodiments, non-peptide linkers used herein include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido] hexanoate (Pierce Chem. Co., Cat #21651G); (iv) Sulfo-LC-SPDP (sulfosuccinimidyl 6 [3-(2-pyridyldithio)-propianamide] hexanoate (Pierce Chem. Co. Cat. #2165-G); and (v) sulfo-NHS (N-hydroxysulfo-succinimide: Pierce Chem. Co., Cat. #24510) conjugated to EDC.

The linkers described above contain components that have different attributes, thus may lead to the agents that comprise IL-22 (such as IL-22 fusion protein, IL-22 dimer) with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form fusion protein with increased stability. Disulfide linkages, are in general, less stable than other linkages because the disulfide linkage is cleaved in vitro, resulting in less antibody fusion protein available. Sulfo-NHS, in particular, can enhance the stability of carbodiimide couplings. Carbodiimide couplings (such as EDC) when used in conjunction with sulfo-NHS, forms esters that are more resistant to hydrolysis than the carbodiimide coupling reaction alone.

### Immunosuppressive Therapy

The immunosuppressive therapy described in this application can be any immunosuppressive drugs in some embodiments is a corticosteroid drug.

In some embodiments, the corticosteroid drug is a systemic corticosteroid.

In some embodiments, the corticosteroid drug is administered at a frequency and dosage that is routinely or often used in treating GvHD. In some embodiments, the corticosteroid is administered daily. In some embodiments, the corticosteroid is administered weekly. In some embodiments, the corticosteroid is administered both during the cycle of administering the IL-22 (e.g., the IL-22 dimer) and the rest period.

In some embodiments, the corticosteroid drug is a prednisone or an equivalent. In some embodiments, the corticosteroid drug is selected from the group consisting of prednisone, betamethasone, prednisolone, triamcinolone, methylprednisolone, and dexamethasone. In some embodiments, the corticosteroid drug is a prednisone. In some embodiments, the prednisone or the equivalent is administered (e.g., intravenously administered) at a dose of no more than about 2 mg/kg for a human being or a comparable dose for an individual who is not a human being. In some embodiments, the prednisone is administered daily.

In some embodiments, the prednisone or the equivalent (such as methylprednisolone) is administered at a dose of about 0.2 mg/kg/day to about 2 mg/kg/day (such as about 0.25 mg/kg/day to about 2 mg/kg/day, such as 0.5 mg/kg/day to about 2 mg/kg/day, such as 1 mg/kg/day to about 2 mg/kg/day) for a human being or a comparable dose for an individual who is not a human being. In some embodiments, the prednisone or the equivalent (such as methylprednisolone) is administered in divided doses.

In some embodiments, the prednisone or the equivalent (such as methylprednisolone) is administered at a dose of about 2 mg/kg/day for a human being or a comparable dose for an individual who is not a human being for at least one, two or three consecutive days after the first administration of the IL-22 dimer. In some embodiments, the prednisone or the equivalent (such as methylprednisolone) is administered at a dose of at least about 0.2 mg/kg/day (such as at least about 0.25 mg/kg/day) for a human being or a comparable dose for an individual who is not a human being for at least about one, two, three or four weeks after the first administration of the IL-22 dimer.

In some embodiments, the method further provides administering a second corticosteroid drug. In some embodiments, the second corticosteroid drug is a poorly absorbable corticosteroid (such as budesonide or beclomethasone). In some embodiments, the second corticosteroid drug is administered simultaneously with the prednisone or the equivalent (such as methylprednisolone).

### GvHD

As used herein, the term “graft vs. host disease” or “GvHD” refers to a condition, including acute and chronic, resulting from transplanted (graft) cell effects on host cells and tissues resulting from GVH. In other words, donor immune cells infused within the graft or donor immune cells that develop from the stem cells, may see the patient's (host) cells as foreign and turn against them with an immune response. As examples, patients who have had a blood or marrow transplant from someone else are at risk of having acute GvHD. Even donors who are HLA-matched with the recipient can cause GvHD because the donor cells can potentially also make an immune response against minor antigen differences in the recipient.

Acute graft-versus-host disease (aGvHD) is specifically a disorder caused by donor immune cells in patients who have had an allogeneic marrow or blood cell transplantation. The most commonly affected tissues are skin intestine and liver. In severe cases, GvHD can cause blistering in the skin or excessive diarrhea and wasting. Also, inflammation caused by donor immune cells in the liver can cause obstruction that causes jaundice. Other tissues such as lung and thymus may also become affected. The diagnosis is usually confirmed by looking at a small piece of skin, liver, stomach or intestine with a microscope for observation of specific inflammatory characteristics. In severe cases, the liver does not function properly to eliminate waste products from the body. Acute GvHD usually begins during the first 3 months after the transplant. In some cases, it can persist, come back or begin more than 3 months after the transplant. In some embodiments, prednisone and/or other immunosuppressive medications are used to treat acute graft-versus-host disease. In some embodiments, other immunosuppressive medications are used if treatment with prednisone is not successful, even though a large proportion of patients is refractory to immunosuppressive medication and die.

Patients who have had acute GvHD and survive have a greater risk of developing chronic GvHD. Older patients, patients who received a peripheral blood (instead of bone marrow) transplant, and patients who had a mismatched or unrelated donor have a greater risk of chronic GvHD. Chronic GvHD usually begins later after transplant and lasts longer than acute GvHD. Patients with Chronic GvHD may present with a wide variety of symptoms. Skin rash and/or mouth sores are among the most common initial signs of the disease. Unlike acute GvHD, chronic GvHD can cause damage in the glands that produce tears in the eyes and saliva in the mouth resulting in dry eyes or a dry mouth. Patients may have mouth ulcers causing pain while eating, skin rashes, or liver inflammation. Chronic GvHD can also cause many other problems. One such problem is formation of scar tissue in the skin (cutaneous sclerosis) and joints. Another such problem is chronic damage to air passages in the lungs (bronchiolitis obliterans syndrome). Prednisone or other similar anti-inflammatory or immunosuppressive medications are used to treat chronic graft-versus-host disease. Other immunosuppressive medications can be used if treatment with prednisone is not successful. Just as in acute GvHD a large proportion of patients is not cured from chronic GvHD.

In some embodiments, the GvHD is acute GvHD (aGvHD). In some embodiments, the acute GvHD is newly diagnosed acute GvHD. In some embodiments, the acute GvHD is classic acute GvHD. In some embodiments, the acute GvHD is persistent, recurrent or late onset acute GvHD.

In some embodiments, the GvHD is chronic GvHD. In some embodiments, the chronic GvHD is classic chronic GvHD.

In some embodiments, the GvHD is an “acute on chronic” GvHD (i.e., with overlap syndrome).

In some embodiments, the GvHD is a gastrointestinal GvHD (GI GvHD). In some embodiments, the GI GvHD is lower GI GvHD. In some embodiments, the GI GvHD is upper GI GvHD. In some embodiments, the GI GvHD (such as lower GI GvHD) is selected from the group consisting of Grade II, Grade III and Grade IV lower GI GvHD. In some embodiments, the grading of GvHD (such as aGvHD) is based on International Bone Marrow Transplant Registry (IBMTR) criteria.

### Patient Population

In some embodiments, the individual is a mammal. In some embodiments, the individual is a human.

In some embodiments, the individual has acute GvHD (aGvHD) (such as newly diagnosed acute GvHD, such as classic acute GvHD, such as persistent, recurrent or late onset acute GvHD.)

In some embodiments, the individual has chronic GvHD (such as classic chronic GvHD).

In some embodiments, the individual has “acute on chronic” GvHD (i.e., with overlap syndrome).

In some embodiments, the individual has gastrointestinal GvHD (GI GvHD). In some embodiments, the individual has lower GI GvHD. In some embodiments, the individual has upper GI GvHD. In some embodiments, the individual has GI GvHD (such as lower GI GvHD) selected from the group consisting of Grade II, Grade III and Grade IV lower GI GvHD.

In some embodiments, the individual is female. In some embodiments, the individual is male.

In some embodiments, the individual does not have an infection at the time of initiating the treatment and/or during the treatment. In some embodiments, the individual has an infection at the time of initiation of the treatment and/or during the treatment. In some embodiments, the infection is a CMV infection.

In some embodiments, the individual has not been subject to a corticosteroid therapy for the GvHD (such as aGvHD) prior to the treatment. In some embodiments, the individual has not been subject to a corticosteroid therapy for the GvHD (such as aGvHD) prior to the treatment for more than about 14, 10, 7, 6, 5, 4, 3, 2, or 1 day.

In some embodiments, the individual has an Ann Arbor score of 3, 2, and/or 1 prior to the treatment. In some embodiments, the individual has an Ann Arbor score of 3. In some embodiments, the individual has an Ann Arbor score of 2. In some embodiments, the individual has an Ann Arbor score of 1.

In some embodiments, the fecal microbiota of the individual is characterized with a PC2 score of no more than 0.5 (for example, no more than 0.45, 0.4, 0.35, 0.3, 0.25, 0.2, 0.15, 0.1, 0.05, 0) based upon analysis of bacterial 16S rRNA sequencing of the fecal sample.

In some embodiments, the individual has a gut microbiota profile characterized with enriched Lachnospiraceae and/or Ruminococcaceae. See, for example, Oki et al. BMC Microbiology (2016) 16:284. In some embodiments, the individual has a gut microbiota profile characterized with enriched Lachnospiraceae. In some embodiments, the individual has a gut microbiota profile characterized with abundant Lachnospiraceae to an extent of about 50%, 60%, 70%, 80%, 90%, 100%, 110%, 120%, 130%, 140% or 150% as a healthy individual (or average of healthy individuals) of the same race/ethnicity and/or in the same region. In some embodiments, the individual has a gut microbiota profile characterized with enriched Ruminococcaceae. In some embodiments, the individual has a gut microbiota profile characterized with abundant Ruminococcaceae to an extent of about 50%, 60%, 70%, 80%, 90%, 100%, 110%, 120%, 130%, 140% or 150% as a healthy individual (or average of healthy individuals) of the same race/ethnicity and/or in the same region.

In some embodiments, the individual is resistant to a prior therapy (such as an immunosuppressive therapy).

### Gut Microbiota

In some embodiments, the abundance of the gut microbiota composition in the individual is increased after the treatment. For example, in some embodiments, the abundance of the gut microbiota composition is increased by at least about 10%, 20%, 30%, 40%, 50%, 60%, 70%, 80%, 90% as compared to the abundance before treatment. The abundance of the gut microbiota composition can be assessed as a whole or on a specific bacterial family. For example, the abundance of the gut microbiota composition can be assessed for Prevotellaceae, Bacteroidaceae, Lachnospiraceae, Ruminococcaceae, and/or Bifidobacteriaceae.

In some embodiments, the diversity of the gut microbiota composition in the individual is increased after the treatment. For example, in some embodiments, the diversity of the gut microbiota composition is increased by at least about 10%, 20%, 30%, 40%, 50%, 60%, 70%, 80%, 90% as compared to the diversity before treatment. The diversity of the gut microbiota composition can be assessed as a whole or on a specific bacterial family. For example, the diversity of the gut microbiota composition can be assessed for Prevotellaceae, Bacteroidaceae, Lachnospiraceae, Ruminococcaceae, and/or Bifidobacteriaceae.

The abundance and/or diversity of the gut microbiota can be characterized by assessing the fetal microbiota of the individual. For example, the abundance and/or diversity of the gut microbiota composition can be assessed based upon analysis of bacterial 16S rRNA sequencing of the fecal sample.

### Dosing and Method of Administering

The dose of the agent that comprises an IL-22 domain (such as an IL-22 dimer) administered to an individual (e.g., a human) in methods described herein may vary with the particular agent, the method of administration, and the type and stage of the GvHD being treated. The amount should be sufficient to produce a desirable response, such as a therapeutic or prophylactic response against GvHD. In some embodiments, the amount of the agent that comprises an IL-22 domain (such as an IL-22 dimer) in the composition is below the level that induces an intolerable toxicological when the agent is administered to the individual according to any of the methods described herein.

In some embodiments, the amount of the agent that comprises an IL-22 domain (such as the IL-22 dimer described herein) for each administration is about 1 μg/kg to about 1 mg/kg (such as about 2 μg/kg to about 500 μg/kg, such as about 2 μg/kg to about 250 μg/kg, such as about 2 μg/kg to about 200 μg/kg, such as about 2 μg/kg to about 100 μg/kg, such as about 2 μg/kg to about 50 μg/kg) for a human being or a comparable dose for an individual who is not a human being. In some embodiments, the amount of the IL-22 dimer for each administration is about 2 μg/kg to about 200 μg/kg for a human being or a comparable dose for an individual who is not a human being. In some embodiments, the amount of the IL-22 dimer for each administration is about 5 μg/kg to about 80 μg/kg, or about 10 μg/kg to about 45 μg/kg (e.g., about 10 μg/kg, 30 μg/kg, or 45 μg/kg) for a human being or a comparable dose for an individual who is not a human being. In some embodiments, the agent is an IL-22 dimer and the amount of the IL-22 dimer for each administration is about 45 μg/kg for a human being or a comparable dose for an individual who is not a human being. The comparable dose for an individual that is not a human can be calculated, for example, based upon guidance provided in Nair, A. B., & Jacob, S. (2016). A simple practice guide for dose conversion between animals and human. Journal of basic and clinical pharmacy, 7(2), 27.

In some embodiments, the agent that comprises an IL-22 domain (such as the IL-22 dimer described herein) is administered at least at a frequency that is about once every one to three times of its serum half-life (such as about twice of its serum half-life). For examples, if the agent has a serum half-life of about 24 hours. Exemplary frequencies include at least about once a day, once every two days, or once every three days. In some embodiments, the agent (such as the IL-22 dimer) is administered at least about or about weekly (for example, daily, weekly, or once every two, three, four, five or six days). In some embodiments, the agent (such as the IL-22 dimer) is administered no more than about once every two days (such as no more than about once every three, four, five, six days, or weekly, or bi-weekly).

In some embodiments, the agent that comprises an IL-22 domain (such as the IL-22 dimer described herein) is administered for a period of at least about or about one, two, three or four weeks at a frequency described herein (such as weekly). In some embodiments the agent (such as the IL-22 dimer) is administered for a period of no more than about six months, five months, four months, three months, two months, one month, or four weeks at a frequency described herein (such as weekly).

In some embodiments, the agent that comprises an IL-22 domain (such as the IL-22 dimer described herein) is administered for at least two cycles, wherein each of the at least two cycles comprises administering the agent (such as the IL-22 dimer described herein) at a frequency described herein (such as weekly) for at least about two weeks (such as about four weeks). In some embodiments, two consecutive cycles of the at least two cycles are separated by a rest period of at least about or about one week (such as at least about or about two, three, or four weeks). In some embodiments, at least one (such as two, three, four or more) of the at least two cycles comprises administering the IL-22 dimer no more than about six months, five months, four months, three months, two months, one month, or four weeks at a frequency described herein (such as weekly). In some embodiments, each of the at least two cycles comprises administering the IL-22 dimer no more than about six months, five months, four months, three months, two months, one month, or four weeks at a frequency described herein (such as weekly). In some embodiments, the rest period between two consecutive cycles varies. In some embodiments, the rest period between two consecutive cycles remains same.

Agents that comprises an IL-22 domain (such as IL-22 dimer as described herein) or suppressive agents described herein can be administered to an individual (such as a human) via various routes, including, for example, intravenous, intra-arterial, intraperitoneal, intrapulmonary, oral, inhalation, intravesicular, intramuscular, intra-tracheal, subcutaneous, intraocular, intrathecal, transmucosal, and transdermal. In some embodiments, the agent that comprises an IL-22 domain (such as IL-22 dimer) and/or the suppressive agent are administered intravenously.

Benefits from using agents comprising IL-22 and methods described herein include prophylactic protection and disease recovery of cells from GVHD including but not limited to cells of the small intestine, large intestine and liver. See WO2015070077, which is incorporated herein by its entirety.

In some embodiments, the number of ISC or Paneth cells are increased after treatment. In some embodiments, the number of ISC or Paneth cells are increased by at least about 5%, 10%, 15%, 20%, 25%, 30%, 35%, 40%, 45%, or 50% after treatment. Number of ISC or Paneth cells can be indicated by measuring the GI expression of molecules involved with ISC and Paneth cell function and kinetics of epithelial renewal. For example, individuals can be evaluated for the kinetics of epithelial regeneration from Lgr5+ cells in both the small and LI, and specific attention will be paid in the SI to the kinetics of Paneth cell renewal. In addition, RNA and protein expression of ISC genes (e.g., Lgr5, BMI-1, Hopx, mTert, Lrigl), Paneth cell genes involved with ISC function (e.g., Wnt3, EGF) by quantitative (q) PCR and western blot in samples from individuals.

In some embodiments, GI epithelial renewal is promoted (such as increased by at least about 5%, 10%, 15%, 20%, 25%, 30%, 35%, 40%, 45%, or 50%) after treatment. Evaluating GI epithelial renewal after treatment with may be done by evaluating for damage to the ISC compartment in individuals. For example, Lgr5 expression as a marker of ISCs supported by the in vivo and ex vivo stem cell function of Lgr5+ cells. An alternative approach to stem cell phenotyping such as assessment of CD44+CD166 CD24″ cells. Paneth cells make up the ISC niche in the small intestine (SI) but are largely absent in the large intestine (LI), where Wnt signals are thought to be provided to the ISCs by niche-supporting kit+ cells. In some embodiments, Paneth cells in the SI are measured to evaluate GI epithelial renewal. In some embodiments, cKit+ LI niche cells are measured to evaluate GI epithelial renewal.

### Methods of Identifying or Selecting an Individual for Treatment

The present application also provides a method of selecting or identifying an individual having GvHD for treatment as described herein.

In some embodiments, there is provided a method of identifying/selecting an individual for a treatment of graft verse host disease (GvHD) in the individual comprising: a) evaluating intestinal microbiome in the individual; and b) selecting an individual for treatment based upon: i) the individual has a gut microbiota profile characterized with enriched Lachnospiraceae and/or Ruminococcaceae; ii) the fecal microbiota of the individual is characterized with a PC2 score of no more than 0.5 (for example, no more than 0.45, 0.4, 0.35, 0.3, 0.25, 0.2, 0.15, 0.1, 0.05, 0) based upon analysis of bacterial 16S rRNA sequencing of the fecal sample; or iii) the individual has an Ann Arbor score of 3, wherein the treatment comprises: i) an effective amount of an IL-22 dimer comprising two monomeric subunits, wherein each monomeric subunit comprises an IL-22 domain and an Fc domain; and ii) an immunosuppressive therapy. In some embodiments, the fecal microbiota of the individual prior to the treatment is characterized with a PC2 score of no more than 0 based upon analysis of bacterial 16S rRNA sequencing of the fecal sample.

In some embodiments, there is provided a method of selecting or identifying an individual having GvHD for treatment with an agent described herein (such as an IL-22 dimer), wherein the method comprises: assessing Ann Arbor score, and selecting or recommending the individual for treatment based upon the Ann Arbor score. In some embodiments, the method comprises selecting or recommending the individual for treatment when the individual has an Ann Arbor score of 3. In some embodiments, the method comprises selecting or recommending the individual for treatment when the individual has an Ann Arbor score of 2. In some embodiments, the method comprises selecting or recommending the individual for treatment when the individual has an Ann Arbor score of 1. In some embodiments, the method further comprises administering an effective amount of an IL-22 dimer comprising two monomeric subunits to the selected individual, wherein each monomeric subunit comprises an IL-22 domain and an Fc domain.

In some embodiments, there is provided a method of selecting or identifying an individual having GvHD for treatment with an agent described herein (such as an IL-22 dimer), wherein the method comprises: assessing the diversity and/or abundance of the gut microbiota in the individual, and selecting or recommending the individual for treatment based upon the diversity and/or abundance of gut microbiota. In some embodiments, the method comprises selecting or recommending an individual for treatment based upon the fecal microbiota of the individual having a PC2 score of no more than 0.5 (for example, no more than 0.45, 0.4, 0.35, 0.3, 0.25, 0.2, 0.15, 0.1, 0.05, 0) based upon analysis of bacterial 16S rRNA sequencing of the fecal sample. In some embodiments, the method further comprises administering an effective amount of an IL-22 dimer comprising two monomeric subunits to the selected individual, wherein each monomeric subunit comprises an IL-22 domain and an Fc domain.

In some embodiments, there is provided a method of selecting or identifying an individual having GvHD for treatment with an agent described herein (such as an IL-22 dimer), wherein the method comprises: assessing gut microbiota profile in the individual, and selecting or recommending the individual for treatment based upon a gut microbiota profile characterized with enriched Lachnospiraceae and/or Ruminococcaceae in gut microbiota in the individual. In some embodiments, the individual has a gut microbiota profile characterized with enriched Lachnospiraceae. In some embodiments, the individual has a gut microbiota profile characterized with abundant Lachnospiraceae to an extent of about 50%, 60%, 70%, 80%, 90%, 100%, 110%, 120%, 130%, 140% or 150% as a healthy individual (or average of healthy individuals) of the same race/ethnicity and/or in the same region. In some embodiments, the individual has a gut microbiota profile characterized with enriched Ruminococcaceae. In some embodiments, the individual has a gut microbiota profile characterized with abundant Ruminococcaceae to an extent of about 50%, 60%, 70%, 80%, 90%, 100%, 110%, 120%, 130%, 140% or 150% as a healthy individual (or average of healthy individuals) of the same race/ethnicity and/or in the same region. In some embodiments, the method further comprises administering an effective amount of an IL-22 dimer comprising two monomeric subunits to the selected individual, wherein each monomeric subunit comprises an IL-22 domain and an Fc domain.

In some embodiments, the method comprises administering an agent comprising IL-22 (such as an IL-22 dimer as described herein) for at least two cycles, wherein each of the at least two cycles comprises administering the IL-22 dimer at least about once a week for a period of at least two weeks, wherein two consecutive cycles of the at least two cycles are separated by a rest period of at least about one week. In some embodiments, the IL-22 domain comprises a recombinant IL-22. In some embodiments, said recombinant IL-22 is a human IL-22. In some embodiments, the IL-22 dimer comprises the amino acid sequence of any one of SEQ ID NOS: 6-9. In some embodiments, the IL-22 dimer comprises the amino acid sequence of SEQ ID NO: 6. In some embodiment, the amount of the IL-22 dimer for each administration is about 1 μg/kg to about 500 μg/kg for a human being or a comparable dose for an individual who is not a human being. In some embodiments, the amount of the IL-22 dimer for each administration is about 45 μg/kg for a human being or a comparable dose for an individual who is not a human being. In some embodiments, the IL-22 dimer is administered intravenously.

### Kits, Medicines and Compositions

The present application also provides kits, medicines, compositions, and unit dosage forms for use in any of the methods described herein.

In some embodiments, there is provided a kit comprising (a) an effective amount of IL-22 (such as an IL-22 dimer comprising two monomeric subunits, wherein each monomeric subunit comprises an IL-22 domain and an Fc domain); and b) an immunosuppressive therapy (such as an immunosuppressive agent, such as a corticosteroid, such as a systemic corticosteroid). In some embodiments, the kit further comprises instructions for use in accordance with any of the methods described herein. In some embodiments, the instructions comprise information relating to dosage and/or dosing schedule of administration for the intended treatment. In some embodiments the instructions provide that the IL-22 (e.g., the IL-22 dimer described herein) is administered for at least two cycles, wherein each of the at least two cycles comprises administering the IL-22 for a period of at least two weeks (e.g., for a period of at least four weeks), wherein two consecutive cycles of the at least two cycles are separated by a rest period of at least about one week (e.g., a rest period of at least about two weeks, three weeks, or four weeks) . . . . The IL-22 and the immunosuppressive therapy can be present in separate containers or in a single container. For example, the kit may comprise one distinct composition or two or more compositions wherein one composition comprises IL-22 (such as IL-22 dimer) and one composition comprises the immunosuppressive therapy (such as a corticosteroid).

In some embodiments, the kit further comprises an agent for assessing the Ann Arbor score of the individual. In some embodiments, the kit further comprises an agent for assessing the diversity and/or abundance of the gut microbiota in the individual. In some embodiments, the kit further comprises an agent for assessing the composition of Lachnospiraceae and/or Ruminococcaceae in gut microbiota in the individual. In some embodiments, the kit further comprises an agent for assessing presence of an infection (such as an active infection that is deemed as uncontrolled infection, such as a CMV infection) in the individual.

The kits of the invention are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The kit described herein may further comprise instructions that include descriptions of selecting an individual suitable or treatment. In some embodiments, the instructions provide that the individual has an Ann Arbor score of 3, 2, and/or 1 prior to the treatment. In some embodiments, the instructions provide that, prior to the treatment: a) the fecal microbiota of the individual is characterized with a PC2 score of no more than 0.5 (such as no more than 0) based upon analysis of bacterial 16S rRNA sequencing of the fecal sample; and/or ii) the individual has a gut microbiota profile characterized with enriched Lachnospiraceae and Ruminococcaceae. In some embodiments, the instructions provide that the individual has an infection.

Instructions supplied in the kits of the invention are typically written instructions on a label or package insert (e.g., a paper sheet included in the kit), but machine-readable instructions (e.g., instructions carried on a magnetic or optical storage disk) are also acceptable.

The instructions generally also include information as to other aspects such as route of administration for the intended treatment. For example, kits may be provided that contain sufficient dosages of the IL-22 (such as IL-22 dimer) and/or the immunosuppressive therapy (such as a corticosteroid) as disclosed herein to provide effective treatment of an individual for any period of time, such as any of about 1, 2, 3, 4, 5, 6, or 7 days, or about 1, 2, 3, 4, 5, or 6 week, or about 1, 2, 3, 4, 5, 6, 7, 8, 9, 10, 11, or 12 months, or 1, 2, 3, 4, 5, 6 years. Kits may also include multiple unit doses of IL-22 (such as IL-22 dimer) and/or the immunosuppressive therapy (such as a corticosteroid) and pharmaceutical compositions and instructions for use and packaged in quantities sufficient for storage and use in pharmacies, for example, hospital pharmacies and compounding pharmacies.

Also provided are medicines, compositions, and unit dosage forms useful for the methods described herein. In some embodiments, there is provided a medicine (or composition) for use in treating GvHD comprising (a) an effective amount of IL-22 (such as an IL-22 dimer comprising two monomeric subunits, wherein each monomeric subunit comprises an IL-22 domain and an Fc domain); and b) an immunosuppressive therapy (such as an immunosuppressive agent, such as a corticosteroid, such as a systemic corticosteroid).

### EXEMPLARY EMBODIMENT

Embodiment 1. A method of treating graft versus host disease (GvHD) in an individual, comprising administering to the individual: a) an effective amount of an IL-22 dimer comprising two monomeric subunits, wherein each monomeric subunit comprises an IL-22 domain and an Fc domain; and b) an immunosuppressive therapy, wherein the IL-22 dimer is administered for at least two cycles, wherein each of the at least two cycles comprises administering the IL-22 dimer for a period of at least two weeks, wherein two consecutive cycles of the at least two cycles are separated by a rest period of at least about one week.

Embodiment 2. The method of embodiment 1, wherein the IL-22 dimer is administered at a frequency of at least about once a week during each of the at least two cycles.

Embodiment 3. The method of embodiment 1 or embodiment 2, wherein at least one of the at least two cycles comprises administering the IL-22 dimer at least about once a week for at least about four weeks.

Embodiment 4. The method of embodiment 3, wherein each of the at least two cycles comprises administering the IL-22 dimer at least once a week for at least about four weeks.

Embodiment 5. The method of any one of embodiments 1-4, wherein at least one of the at least two cycles comprises administering the IL-22 dimer no more than about three months.

Embodiment 6. The method of embodiment 5, wherein each of the at least two cycles comprises administering the IL-22 dimer no more than about three months.

Embodiment 7. The method of any one of embodiments 1-6, wherein the rest period is about one to four weeks.

Embodiment 8. The method of any one of embodiments 1-6, wherein the rest period is at least about four weeks.

Embodiment 9. The method of embodiment 8, wherein the rest period is no more than three months.

Embodiment 10. The method of any one of embodiments 1-9, wherein the individual has an Ann Arbor score of 1, 2, and/or 3.

Embodiment 11. The method of any one of embodiments 1-10, wherein: a) the fecal microbiota of the individual prior to the treatment is characterized with a PC2 score of no more than 0.5 based upon analysis of bacterial 16S rRNA sequencing of the fecal sample; or b) the individual has a gut microbiota profile characterized with enriched Lachnospiraceae and Ruminococcaceae.

Embodiment 12. A method of treating graft versus host disease (GvHD) in an individual, comprising administering to the individual: a) an effective amount of an IL-22 dimer comprising two monomeric subunits, wherein each monomeric subunit comprises an IL-22 domain and an Fc domain; and b) an immunosuppressive therapy, wherein the individual has an Ann Arbor score of 3 prior to the treatment.

Embodiment 13. The method of embodiment 12, wherein the method further comprises assessing Ann Arbor score of the individual.

Embodiment 14. The method of embodiment 12 or embodiment 13, wherein the method further comprises selecting the individual for the treatment based upon the individual having an Ann Arbor score of 3.

Embodiment 15. A method of treating graft versus host disease (GvHD) in an individual, comprising administering to the individual: a) an effective amount of an IL-22 dimer comprising two monomeric subunits, wherein each monomeric subunit comprises an IL-22 domain and an Fc domain; and b) an immunosuppressive therapy, and wherein prior to the treatment: i) the fecal microbiota of the individual is characterized with a PC2 score of no more than 0.5 based upon analysis of bacterial 16S rRNA sequencing of the fecal sample; or ii) the individual has a gut microbiota profile characterized with enriched Lachnospiraceae and Ruminococcaceae.

Embodiment 16. The method of embodiment 15, wherein the method further comprises assessing bacterial 16s rRNA gene sequence in fecal sample of the individual.

Embodiment 17. The method of embodiment 15 or embodiment 16, wherein the method further comprises selecting the individual for the treatment based upon the fecal microbiota of the individual having a PC2 score of no more than 0.5 based upon analysis of bacterial 16S rRNA sequencing of the fecal sample.

Embodiment 18. A method of treating graft versus host disease (GvHD) in an individual, comprising administering to the individual: a) an effective amount of an IL-22 dimer comprising two monomeric subunits, wherein each monomeric subunit comprises an IL-22 domain and an Fc domain; and b) an immunosuppressive therapy, wherein the individual has an infection.

Embodiment 19. The method of embodiment 18, wherein the immunosuppressive therapy comprises a corticosteroid, wherein the corticosteroid is optionally contraindicated for the individual.

Embodiment 20. A method of identifying/selecting an individual for a treatment of graft verse host disease (GvHD) in the individual comprising: a) evaluating intestinal microbiome in the individual; and b) selecting an individual for treatment based upon: i) the individual has a gut microbiota profile characterized with enriched Lachnospiraceae and/or Ruminococcaceae; ii) the fecal microbiota of the individual is characterized with a PC2 score of no more than 0.5 based upon analysis of bacterial 16S rRNA sequencing of the fecal sample; or iii) the individual has an Ann Arbor score of 3, wherein the treatment comprises: i) an effective amount of an IL-22 dimer comprising two monomeric subunits, wherein each monomeric subunit comprises an IL-22 domain and an Fc domain; and ii) an immunosuppressive therapy.

Embodiment 21. The method of embodiment 20, wherein the fecal microbiota of the individual prior to the treatment is characterized with a PC2 score of no more than 0 based upon analysis of bacterial 16S rRNA sequencing of the fecal sample.

Embodiment 22. The method of any one of embodiments 12-21, wherein the method of any one of embodiments 1-19 or the treatment of embodiment 20 or 21 comprises administering the IL-22 dimer for at least two cycles, wherein each of the at least two cycles comprises administering the IL-22 dimer at least about once a week for a period of at least two weeks, wherein two consecutive cycles of the at least two cycles are separated by a rest period of at least about one week.

Embodiment 23. The method of any one of embodiments 1-22, wherein the IL-22 domain comprises a recombinant IL-22.

Embodiment 24. The method of embodiment 23, wherein said recombinant IL-22 is a human IL-22.

Embodiment 25. The method of any one of embodiments 1-24, wherein the IL-22 dimer comprises the amino acid sequence of any one of SEQ ID NOS: 6-9.

Embodiment 26. The method of any one of embodiments 1-25, wherein the IL-22 dimer comprises the amino acid sequence of SEQ ID NO: 6.

Embodiment 27. The method of any one of embodiments 1-26, wherein the amount of the IL-22 dimer for each administration is about 1 μg/kg to about 500 μg/kg for a human being or a comparable dose for an individual who is not a human being.

Embodiment 28. The method of embodiment 27, wherein the amount of the IL-22 dimer for each administration is about 2 μg/kg to about 200 μg/kg for a human being or a comparable dose for an individual who is not a human being.

Embodiment 29. The method of embodiment 28, wherein the amount of the IL-22 dimer for each administration is about 5 μg/kg to about 80 μg/kg for a human being or a comparable dose for an individual who is not a human being.

Embodiment 30. The method of embodiment 29, wherein the amount of the IL-22 dimer for each administration is about 10 μg/kg to about 45 μg/kg (such as about 10 μg/kg, about 30 μg/kg, or about 45 μg/kg) for a human being or a comparable dose for an individual who is not a human being.

Embodiment 31. The method of any one of embodiments 1-30, wherein the IL-22 dimer is administered intravenously.

Embodiment 32. The method of any one of embodiments 1-31, wherein the GvHD is acute GvHD.

Embodiment 33. The method of any one of embodiments 1-32, wherein the GvHD is a gastrointestinal GvHD (GI GvHD).

Embodiment 34. The method of embodiment 33, wherein the GI GvHD is lower GI GvHD.

Embodiment 35. The method of embodiment 34, wherein the lower GI GvHD is selected from the group consisting of Grade II, Grade III and Grade IV lower GI GvHD.

Embodiment 36. The method of any one of embodiments 1-35, wherein the individual has not been subject to corticosteroid for GvHD for a period of three or more days prior to the treatment.

Embodiment 37. The method of any one of embodiments 1-36, wherein the individual does not have an ongoing Cytomegalovirus (CMV) infection immediately prior to the treatment.

Embodiment 38. The method of any one of embodiments 1-37, wherein the individual is a human.

Embodiment 39. The method of any one of embodiments 1-38, wherein the immunosuppressive therapy comprises a corticosteroid drug.

Embodiment 40. The method of embodiment 39, wherein the corticosteroid drug is a systemic corticosteroid.

Embodiment 41. The method of embodiment 39 or embodiment 40, wherein the corticosteroid drug is a prednisone.

Embodiment 42. The method of embodiment 41, wherein the prednisone is administered at a dose of no more than about 2 mg/kg for a human being or a comparable dose for an individual who is not a human being.

Embodiment 43. The method of any one of embodiments 39-42, wherein the corticosteroid drug is administered daily.

### EXAMPLES

The examples below are intended to be purely exemplary of the invention and should therefore not be considered to limit the invention in any way. The following examples and detailed description are offered by way of illustration and not by way of limitation.

### Example 1. A Phase 2 Study of F-652, a Novel Tissue-Targeted Recombinant Human Interleukin-22 (IL-22) Dimer, for Treatment of Newly Diagnosed Acute Lower GI GvHD

F-652 is recombinant IL-22 dimer consisting of two monomeric subunits each comprising a sequence shown in SEQ ID NO: 6. In this study, it was tested if the addition of F-652 to corticosteroids could promote healing of GI tract injury and improve treatment response in patients with lower GI aGvHD.

A 27-patient open-label, single-cohort, multicenter prospective phase 2 study was conducted. Eligible patients were ≥18 years old and had new onset biopsy-proven grade II-IV lower GI aGvHD following an allo-HCT. Patients were treated with four weekly doses of F-652, a recombinant human IL-22 dimer/Fc fusion molecule at a dose of 45 μg/kg intravenously, in combination with standard corticosteroid treatment. Patients were administered the third and fourth doses only after demonstrating a treatment response to the first two doses. Primary endpoints included PK, safety, and day 28 lower GI aGvHD response. Additional endpoints included day 56 treatment response, evaluation of changes in gut microbiota by 16S sequencing, and plasma GvHD biomarkers. The study was powered to distinguish between an unpromising response rate of 35% and a promising response rate of 60%.

The 27 patients (median age 55 years, mostly PBSC recipients) had predominantly stage 2-4 lower GI GvHD (17/27, 63%), with 6/27 (22%) patients having stage 3 and 6/27 (22%) patients having stage 4 disease. All patients had detectable F-652 levels and measurement of CRP levels in a subset of patients confirmed in vivo biologic activity.

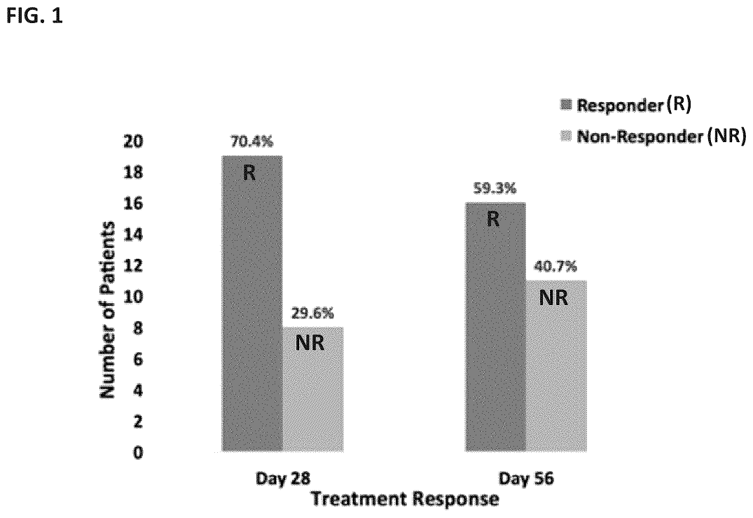

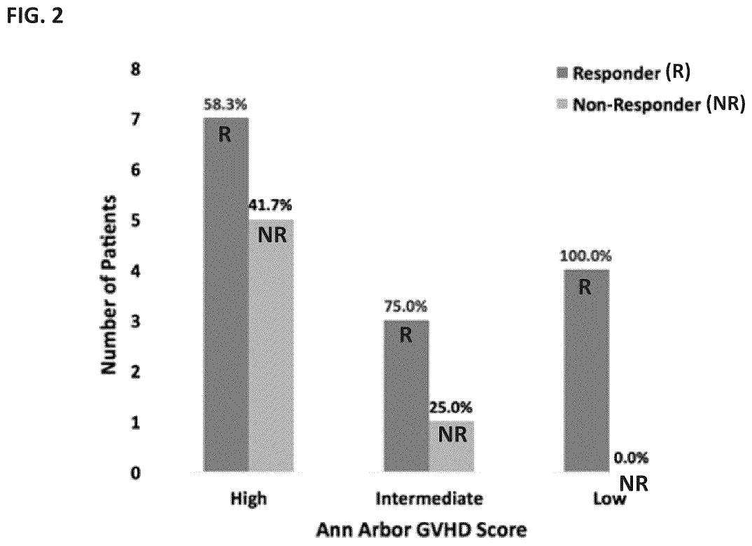

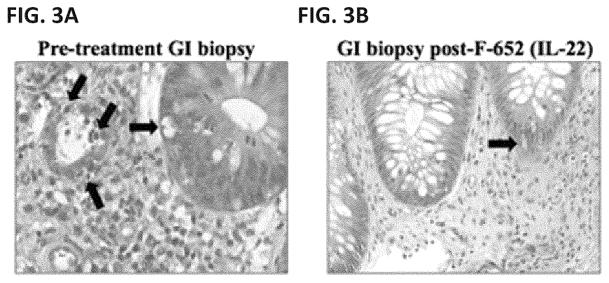

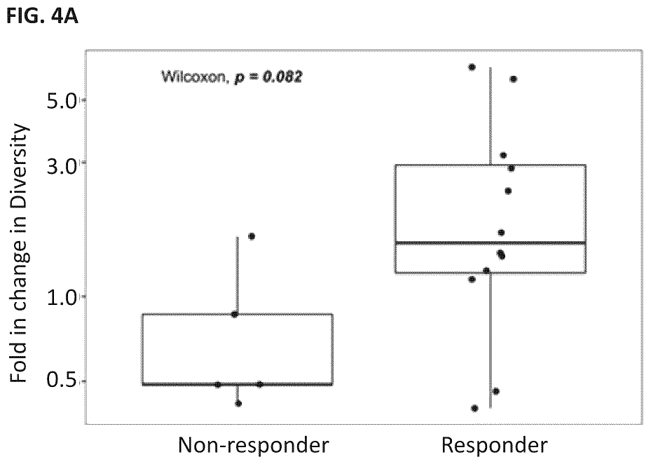

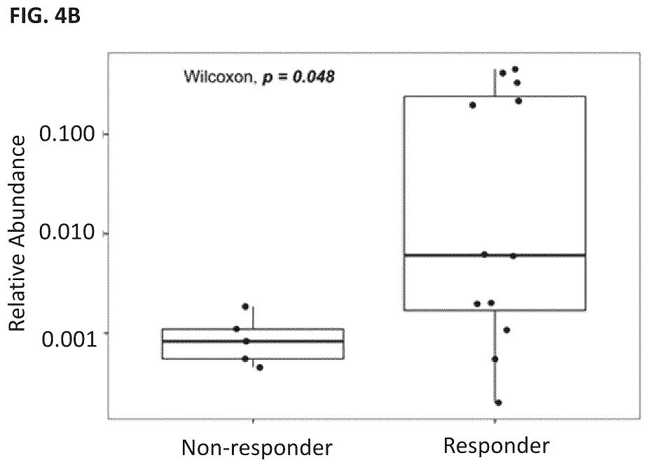

Overall, 19 out of 27 patients achieved a day 28 response (70%, 90% CI: 56-79). See FIG. 1. Response to treatment based on Ann Arbor Risk, evaluable in 20 patients, was 7/12 (58%) with high, 3/4 (75%) with intermediate, and 4/4 (100%) with low risk biomarkers. See FIG. 2. Surprisingly, at day 56, the majority (sixteen) patients remained treatment responders (59%, 90% CI: 45-69). See FIG. 1. Three patients had repeat GI biopsy after treatment and demonstrated improvement in GI epithelial injury. See FIGS. 3A-3B. Additionally, in a subset of 17 patients with evaluable stool samples, microbial diversity and the relative abundance of commensal Blautia were higher in patients with a clinical response to F-652 (p=0.082 and 0.048, respectively.). See FIGS. 4A-4B.

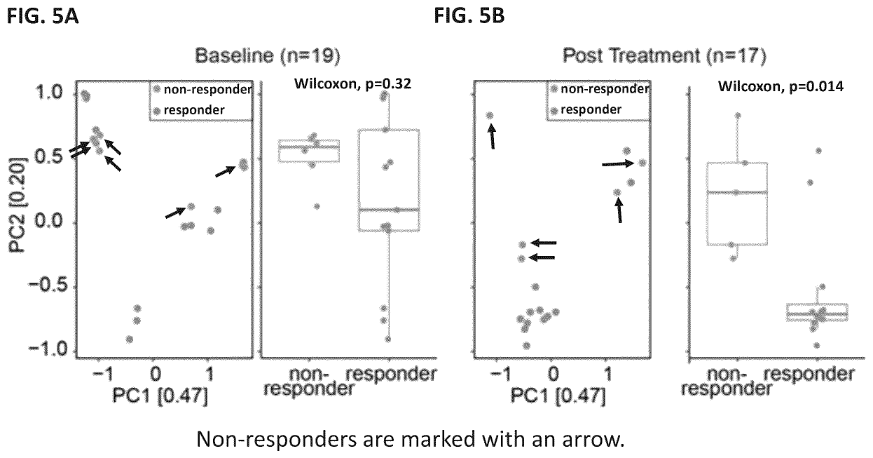

Principal component analysis (PCA) demonstrated that baseline microbiota composition was similar among patients (n=19 patients) whereas the global microbiota composition was significantly different between responders and non-responders (n=17 patients, p=0.01). See FIGS. 5A-5B. Specifically, at the baseline, about 82% of the responders had a PC2 value that is lower than 0.5, and 100% of the responders had a PC2 value that is lower than 0. No such trend is shown in a similar PC1 analysis. See Tables 1 and 2.

TABLE 1

| | |
|---|---|
| Responders (%) | Non-responders (%) |

Cut-off: PC2 = 0.5

| | | | | |
|---|---|---|---|---|
| PC2 < 0.5 | 9/11 | (81.8%) | 2/11 | (18.2%) |
| PC2 > 0.5 | 4/8 | (50%) | 4/8 | (50%) |

Cut-off: PC2 = 0

| | | | | |
|---|---|---|---|---|
| PC2 < 0 | 6/6 | (100%) | 0/6 | (0%) |
| PC2 > 0 | 7/13 | (53.8%) | 6/13 | (46.2%) |

TABLE 2

| | |
|---|---|
| Responders (%) | Non-responders (%) |

Cut-off: PC1 = 1

| | | | | |
|---|---|---|---|---|
| PC1 < 1 | 9/14 | (64.3%) | 5/14 | (35.7%) |
| PC1 > 1 | 4/5 | (80%) | 1/5 | (20%) |

Cut-off: PC1 = 0

| | | | | |
|---|---|---|---|---|
| PC1 < 0 | 7/11 | (63.6%) | 4/11 | (36.4%) |
| PC1 > 0 | 6/8 | (75%) | 2/8 | (25%) |

Cut-off: PC1 = −1

| | | | | |
|---|---|---|---|---|
| PC1 < −1 | 4/8 | (50%) | 4/8 | (50%) |
| PC1 > −1 | 7/9 | (77.8%) | 2/9 | (22.2%) |

Serious TEAEs were observed in 11 patients (40%) including enterocolitis (n=1), pyrexia (n=1), infection (2 sepsis, 1 device-related, 1 pneumonia, 1 sinusitis), musculoskeletal (n=2), and respiratory (n=1). One patient was diagnosed with squamous cell carcinoma 8 months after F-652 treatment.

SEQUENCE TABLE

| SEQ | | |
|---|---|---|
| ID NO | Description | Amino acid sequence |
| 1. | IL-22 | APISSHCRLDKSNFQQPYITNRTFMLAKEASLADNNTDVRLIGE |
| | | KLFHGVSMSERCYLMKQVLNFTLEEVLFPQSDRFQPYMQEVVP |
| | | FLARLSNRLSTCHIEGDDLHIQRNVQKLKDTVKKLGESGEIKAI |
| | | GELDLLFMSLRNACI |
| 2. | IgG2 Fc | VECPPCPAPPVAGPSVFLFPPKPKDTLMISRTPEVTCVVVDVSHE |
| | | DPEVQFNWYVDGVEVHNAKTKPREEQFNSTFRVVSVLTVVHQ |
| | | DWLNGKEYKCKVSNKGLPASIEKHSKTKGQPREPQVYTLPPSR |
| | | EEMTKNQVSLTCLVKGFYPSDIAVEWESNGQPENNYKTTPPML |
| | | DSDGSFFLYSKLTVDKSRWQQGNVFSCSVMHEALHNHYTQKS |
| | | LSLSPGK |
| 3. | IgG2 Fc | ERKCCVECPPCPAPPVAGPSVFLFPPKPKDTLMISRTPEVTCVVV |
| | | DVSHEDPEVQFNWYVDGVEVHNAKTKPREEQFNSTFRVVSVL |
| | | TVVHQDWLNGKEYKCKVSNKGLPAPIEKTISKTKGQPREPQVY |
| | | TLPPSREEMTKNQVSLTCLVKGFYPSDISVEWESNGQPENNYKT |
| | | TPPMLDSDGSFFLYSKLTVDKSRWQQGNVFSCSVMHEALHNH |
| | | YTQKSLSLSPGK |
| 4. | Peptide linker | GSGGGSGGGGSGGGGS |
| 5. | Peptide linker | ASTKGP |
| 6. | IL22-linker-IgG2 | APISSHCRLDKSNFQQPYITNRTFMLAKEASLADNNTDVRLIGE |
| | Fc | KLFHGVSMSERCYLMKQVLNFTLEEVLFPQSDRFQPYMQEVVP |
| | | FLARLSNRLSTCHIEGDDLHIQRNVQKLKDTVKKLGESGEIKAI |
| | | GELDLLFMSLRNACIGSGGGSGGGGSGGGGSVECPPCPAPPVA |
| | | GPSVFLFPPKPKDTLMISRTPEVTCVVVDVSHEDPEVQFNWYV |
| | | DGVEVHNAKTKPREEQFNSTFRVVSVLTVVHQDWLNGKEYKC |
| | | KVSNKGLPASIEKTISKTKGQPREPQVYTLPPSREEMTKNQVSL |
| | | TCLVKGFYPSDIAVEWESNGQPENNYKTTPPMLDSDGSFFLYS |
| | | KLTVDKSRWQQGNVFSCSVMHEALHNHYTQKSLSLSPGK |
| 7. | IL-22-linker- | APISSHCRLDKSNFQQPYITNRTFMLAKEASLADNNTDVRLIGE |
| | IgG2 Fc | KLFHGVSMSERCYLMKQVLNFTLEEVLFPQSDRFQPYMQEVVP |
| | | FLARLSNRLSTCHIEGDDLHIQRNVQKLKDTVKKLGESGEIKAI |
| | | GELDLLFMSLRNACIASTKGPVECPPCPAPPVAGPSVFLFPPKPK |
| | | DTLMISRTPEVTCVVVDVSHEDPEVQFNWYVDGVEVHNAKTK |
| | | PREEQFNSTFRVVSVLTVVHQDWLNGKEYKCKVSNKGLPASIE |
| | | KTISKTKGQPREPQVYTLPPSREEMTKNQVSLTCLVKGFYPSDI |
| | | AVEWESNGQPENNYKTTPPMLDSDGSFFLYSKLTVDKSRWQQ |
| | | GNVFSCSVMHEALHNHYTQKSLSLSPGK |
| 8. | IgG2 Fc-linker- | VECPPCPAPPVAGPSVFLFPPKPKDTLMISRTPEVTCVVVDVSHE |
| | IL-22 | DPEVQFNWYVDGVEVHNAKTKPREEQFNSTFRVVSVLTVVHQ |
| | | DWLNGKEYKCKVSNKGLPASIEKHSKTKGQPREPQVYTLPPSR |
| | | EEMTKNQVSLTCLVKGFYPSDIAVEWESNGQPENNYKTTPPML |
| | | DSDGSFFLYSKLTVDKSRWQQGNVFSCSVMHEALHNHYTQKS |
| | | LSLSPGKGSGGGSGGGGSGGGGSAPISSHCRLDKSNFQQPYITN |
| | | RTFMLAKEASLADNNTDVRLIGEKLFHGVSMSERCYLMKQVL |
| | | NFTLEEVLFPQSDRFQPYMQEVVPFLARLSNRLSTCHIEGDDLHI |
| | | QRNVQKLKDTVKKLGESGEIKAIGELDLLFMSLRNACI |
| 9. | IgG2 Fc-linker- | VECPPCPAPPVAGPSVFLFPPKPKDTLMISRTPEVTCVVVDVSHE |
| | IL-22 | DPEVQFNWYVDGVEVHNAKTKPREEQFNSTFRVVSVLTVVHQ |
| | | DWLNGKEYKCKVSNKGLPASIEKHSKTKGQPREPQVYTLPPSR |
| | | EEMTKNQVSLTCLVKGFYPSDIAVEWESNGQPENNYKTTPPML |
| | | DSDGSFFLYSKLTVDKSRWQQGNVFSCSVMHEALHNHYTQKS |
| | | LSLSPGKASTKGPAPISSHCRLDKSNFQQPYITNRTFMLAKEASL |
| | | ADNNTDVRLIGEKLFHGVSMSERCYLMKQVLNFTLEEVLFPQS |
| | | DRFQPYMQEVVPFLARLSNRLSTCHIEGDDLHIQRNVQKLKDT |
| | | VKKLGESGEIKAIGELDLLFMSLRNACI |
| 10. | IL-22-linker-IL- | APISSHCRLDKSNFQQPYITNRTFMLAKEASLADNNTDVRLIGE |
| | 22 | KLFHGVSMSERCYLMKQVLNFTLEEVLFPQSDRFQPYMQEVVP |
| | | FLARLSNRLSTCHIEGDDLHIQRNVQKLKDTVKKLGESGEIKAI |
| | | GELDLLFMSLRNACIGSGGGSGGGGSGGGGSAPISSHCRLDKSN |
| | | FQQPYITNRTFMLAKEASLADNNTDVRLIGEKLFHGVSMSERC |
| | | YLMKQVLNFTLEEVLFPQSDRFQPYMQEVVPFLARLSNRLSTC |
| | | HIEGDDLHIQRNVQKLKDTVKKLGESGEIKAIGELDLLFMSLRN |
| | | ACI |
| 11. | Peptide linker | (G)ₙ, n >= 1 |
| 12. | Peptide linker | (GS)ₙ, n >= 1 |
| 13. | Peptide linker | (GSGGS)ₙ, n >= 1 |
| 14. | Peptide linker | (GGGGS)ₙ, n >= 1 |
| 15. | Peptide linker | (GGGS)ₙ, n >= 1 |
| 16. | Peptide linker | (GGGGS)ₙ, n is an integer between 1 and 8 |
| 17. | Peptide linker | (GGGGS)₃ |
| 18. | Peptide linker | (GGGGS)₆ |
| 19. | Peptide linker | (GSTSGSGKPGSGEGS)ₙ, n is between 1 and 3 |

1. A method of treating graft versus host disease (GvHD) in an individual, comprising administering to the individual:
a) an effective amount of an IL-22 dimer comprising two monomeric subunits, wherein each monomeric subunit comprises an IL-22 domain and an Fc domain; and
b) an immunosuppressive therapy,
wherein the IL-22 dimer is administered for at least two cycles, wherein each of the at least two cycles comprises administering the IL-22 dimer for a period of at least two weeks, wherein two consecutive cycles of the at least two cycles are separated by a rest period of at least about one week.

2. The method of claim 1, wherein the IL-22 dimer is administered at a frequency of at least about once a week during each of the at least two cycles.

3. The method of claim 1 or claim 2, wherein at least one of the at least two cycles comprises administering the IL-22 dimer at least about once a week for at least about four weeks.

4. The method of claim 3, wherein each of the at least two cycles comprises administering the IL-22 dimer at least once a week for at least about four weeks.

5. The method of any one of claims 1-4, wherein at least one of the at least two cycles comprises administering the IL-22 dimer no more than about three months.

6. The method of claim 5, wherein each of the at least two cycles comprises administering the IL-22 dimer no more than about three months.

7. The method of any one of claims 1-6, wherein the rest period is about one to four weeks.

8. The method of any one of claims 1-6, wherein the rest period is at least about four weeks.

9. The method of claim 8, wherein the rest period is no more than three months.

10. The method of any one of claims 1-9, wherein the individual has an Ann Arbor score of 1, 2, and/or 3.

11. The method of any one of claims 1-10, wherein:
a) the fecal microbiota of the individual prior to the treatment is characterized with a PC2 score of no more than 0.5 based upon analysis of bacterial 16S rRNA sequencing of the fecal sample; or
b) the individual has a gut microbiota profile characterized with enriched Lachnospiraceae and Ruminococcaceae.

12. A method of treating graft versus host disease (GvHD) in an individual, comprising administering to the individual:
a) an effective amount of an IL-22 dimer comprising two monomeric subunits, wherein each monomeric subunit comprises an IL-22 domain and an Fc domain; and
b) an immunosuppressive therapy,
wherein the individual has an Ann Arbor score of 3 prior to the treatment.

13. The method of claim 12, wherein the method further comprises assessing Ann Arbor score of the individual.

14. The method of claim 12 or claim 13, wherein the method further comprises selecting the individual for the treatment based upon the individual having an Ann Arbor score of 3.

15. A method of treating graft versus host disease (GvHD) in an individual, comprising administering to the individual:
a) an effective amount of an IL-22 dimer comprising two monomeric subunits, wherein each monomeric subunit comprises an IL-22 domain and an Fc domain; and
b) an immunosuppressive therapy,
and wherein prior to the treatment:
i) the fecal microbiota of the individual is characterized with a PC2 score of no more than 0.5 based upon analysis of bacterial 16S rRNA sequencing of the fecal sample; or
ii) the individual has a gut microbiota profile characterized with enriched Lachnospiraceae and Ruminococcaceae.

16. The method of claim 15, wherein the method further comprises assessing bacterial 16s rRNA gene sequence in fecal sample of the individual.

17. The method of claim 15 or claim 16, wherein the method further comprises selecting the individual for the treatment based upon the fecal microbiota of the individual having a PC2 score of no more than 0.5 based upon analysis of bacterial 16S rRNA sequencing of the fecal sample.

18. A method of treating graft versus host disease (GvHD) in an individual, comprising administering to the individual:
a) an effective amount of an IL-22 dimer comprising two monomeric subunits, wherein each monomeric subunit comprises an IL-22 domain and an Fc domain; and
b) an immunosuppressive therapy,
wherein the individual has an infection.

19. The method of claim 18, wherein the immunosuppressive therapy comprises a corticosteroid, which is optionally contraindicated for the individual.

20. A method of identifying/selecting an individual for a treatment of graft verse host disease (GvHD) in the individual comprising:
a) evaluating intestinal microbiome in the individual; and
b) selecting an individual for treatment based upon:
i) the individual has a gut microbiota profile characterized with enriched Lachnospiraceae and/or Ruminococcaceae;
ii) the fecal microbiota of the individual is characterized with a PC2 score of no more than 0.5 based upon analysis of bacterial 16S RNA sequencing of the fecal sample; or
iii) the individual has an Ann Arbor score of 3,
wherein the treatment comprises: i) an effective amount of an IL-22 dimer comprising two monomeric subunits, wherein each monomeric subunit comprises an IL-22 domain and an Fc domain; and ii) an immunosuppressive therapy.

21. The method of claim 20, wherein the fecal microbiota of the individual prior to the treatment is characterized with a PC2 score of no more than 0 based upon analysis of bacterial 16S rRNA sequencing of the fecal sample.

22. The method of any one of claims 12-21, wherein the method of any one of claims 1-19 or the treatment of claim 20 or 21 comprises administering the IL-22 dimer for at least two cycles, wherein each of the at least two cycles comprises administering the IL-22 dimer at least about once a week for a period of at least two weeks, wherein two consecutive cycles of the at least two cycles are separated by a rest period of at least about one week.

23. The method of any one of claims 1-22, wherein the IL-22 domain comprises a recombinant IL-22.

24. The method of claim 23, wherein said recombinant IL-22 is a human IL-22.

25. The method of any one of claims 1-24, wherein the IL-22 dimer comprises the amino acid sequence of any one of SEQ ID NOS: 6-9.

26. The method of any one of claims 1-25, wherein the IL-22 dimer comprises the amino acid sequence of SEQ ID NO: 6.

27. The method of any one of claims 1-26, wherein the amount of the IL-22 dimer for each administration is about 1 μg/kg to about 500 μg/kg for a human being or a comparable dose for an individual who is not a human being.

28. The method of claim 27, wherein the amount of the IL-22 dimer for each administration is about 2 μg/kg to about 200 μg/kg for a human being or a comparable dose for an individual who is not a human being.

29. The method of claim 28, wherein the amount of the IL-22 dimer for each administration is about 5 μg/kg to about 80 μg/kg for a human being or a comparable dose for an individual who is not a human being.

30. The method of claim 29, wherein the amount of the IL-22 dimer for each administration is about 10 μg/kg to about 45 μg/kg for a human being or a comparable dose for an individual who is not a human being.

31. The method of any one of claims 1-30, wherein the IL-22 dimer is administered intravenously.

32. The method of any one of claims 1-31, wherein the GvHD is acute GvHD.

33. The method of any one of claims 1-32, wherein the GvHD is a gastrointestinal GvHD (GI GvHD).

34. The method of claim 33, wherein the GI GvHD is lower GI GvHD.

35. The method of claim 34, wherein the lower GI GvHD is selected from the group consisting of Grade II, Grade III and Grade IV lower GI GvHD.

36. The method of any one of claims 1-35, wherein the individual has not been subject to corticosteroid for GvHD for a period of three or more days prior to the treatment.

37. The method of any one of claims 1-36, wherein the individual does not have an ongoing Cytomegalovirus (CMV) infection immediately prior to the treatment.

38. The method of any one of claims 1-37, wherein the individual is a human.

39. The method of any one of claims 1-38, wherein the immunosuppressive therapy comprises a corticosteroid drug.

40. The method of claim 39, wherein the corticosteroid drug is a systemic corticosteroid.

41. The method of claim 39 or claim 40, wherein the corticosteroid drug is a prednisone.

42. The method of claim 41, wherein the prednisone is administered at a dose of no more than about 2 mg/kg for a human being or a comparable dose for an individual who is not a human being.

43. The method of any one of claims 39-42, wherein the corticosteroid drug is administered daily.
